(19) United States
(12) Patent Application Publication
(10) Pub. No.: US 2011/0166112 A1
(43) Pub. Date: Jul. 7, 2011

(54) METHOD FOR STIMULATING PLATELET PRODUCTION

(75) Inventor: Yanzhen Zhang, Sudbury, MA (US)

(73) Assignee: Eisai, Inc., Woodcliff Lake, NJ (US)

(21) Appl. No.: 12/856,003

(22) Filed: Aug. 13, 2010

Related U.S. Application Data
(60) Provisional application No. 61/234,153, filed on Aug. 14, 2009.
(60) Provisional application No. 61/365,479, filed on Jul. 19, 2010.

(51) Int. Cl. A61K 31/573; A61K 31/496; A61P 7/02; A61P 35/00; A61P 1/16; A61P 31/12; A61P 31/04

(52) U.S. Cl. 514/171; 514/253.1

(57) ABSTRACT

Provided are methods for increasing platelet response in a subject at risk for bleeding due at least in part to a low platelet count by administering to a subject with a low platelet count an effective amount of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid.

### RELATED APPLICATIONS

This application is claims priority to U.S. Provisional Patent Application No. 61/234,153, filed Aug. 14, 2009 and U.S. Provisional Patent Application No. 61/365,479, filed Jul. 19, 2010. The contents of the foregoing applications are hereby incorporated in their entirety.

### BACKGROUND OF THE INVENTION

Thrombocytopenia is a potentially serious condition characterized by a deficiency of platelets in the circulatory system. It is associated with an increased risk of bleeding, particularly from small capillaries, resulting in thrombocytopenic purpura. The causes of thrombocytopenia include decreases in platelet production in the bone marrow and decreases in platelet survival in the blood. There are specific disease-related thrombocytopenias, such as immune (idiopathic) thrombocytopenic purpura (ITP) and thrombocytopenias caused by the indirect effect of other diseases on the bone marrow, including malignancies and infections such as hepatitis. Currently, management of thrombocytopenia is primarily based on platelet transfusion. Despite the effectiveness of transfusion, approximately 30% of transfusions are associated with serious complications, including alloimmunization, febrile and allergic reactions, circulatory overload, acute pulmonary injury and bacterial or viral infections. In the 15 to 25% of patients who require repeated platelet transfusions, the platelet response is inadequate due to human leukocyte antigen (HLA) alloimmunization. Therefore, a safe thrombopoietic agent that can lessen or eliminate the need for platelet transfusion would benefit patient health and could significantly lower health-care costs.

### SUMMARY OF THE INVENTION

The present invention is based, at least in part, on the discovery that 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid):

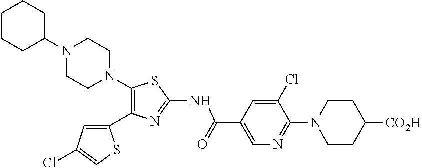

has the ability to increase the platelet response in a rapid manner when compared to conventional drugs, which may be useful in preparing subjects that are at risk of bleeding due to a low platelet count for treatment that can induce bleeding. Accordingly, it is an object of the present invention to provide methods for rapidly increasing the platelet response in a subject in order to prevent bleeding when the subject undergoes a treatment that induces bleeding.

Accordingly, in some aspects, the present invention provides method for rapidly stimulating the platelet response in a subject at risk for bleeding or with active bleeding due at least in part to a low platelet count. The methods generally include administering to the subject an effective amount of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid, wherein the platelet response is increased in less than 14 days.

In some embodiments, the method further provides the step of detecting the platelet response within about 14 days, about 7 days, about 3 days or about 24 hours after administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid.

In some embodiments, the subject has an initial platelet count of less than or about equal to 30,000/mm³ and the platelet response is increased in less than about 7 days to greater than or equal to about 50,0000/mm³.

In some embodiments, the subject has a response rate of at least about 10%, about 25%, about 50% or about 80% over the initial platelet count at about 28 days after administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid.

In some embodiment, the subject has a response rate of at least about 5%, about 10%, about 25%, about 50%, about 70%, about 90% or about 98% over initial platelet count at about 7 days after administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid.

In some embodiments, the subject has a sustained platelet response of at least about 25%, about 30%, about 40%, about 50%, about 75% or about 77% after about 28 days.

In some embodiments, the platelet response is maintained for at least about 7 days, about 14 days, about 28 days, about 2 months, about 3 months, about 4 months, about 5 months, about 6 months or about 7 months. In some embodiments, the platelet response is maintained indefinitely.

In some embodiments, the platelet response is increased by at least about 10%, by about 25%, by about 50% or by about 90% over the initial platelet count.

In some embodiments, the platelet response is increased by between at least about 10,000/mm³ and about 400,000/mm³.

In some embodiments, the effective amount is an effective periodic dose.

In some embodiments, the effective periodic dose is a once daily dose, a twice daily dose, a thrice daily dose, a dose administered every other day, a weekly dose or a monthly dose.

In some embodiments, the effective periodic dose is administered for about 1 month, about 2 months, about 3 months, about 4 months, about 5 months, about 6 months or about 7 months.

In some embodiments, the subject is in need of treatment that may induce bleeding.

In some embodiments, the subject has a low platelet count within at least about 1 month of treatment, within at least about 14 days of treatment, within at least about 7 days of treatment or at least about the time of treatment.

In some embodiments, the treatment comprises a surgery or administration of a therapeutic agent.

In some embodiments, the therapeutic agent comprises chemotherapy, radiation therapy or a combination thereof.

In some embodiment, surgery comprises administration of an anesthetic, administration of an epidural, a biopsy, a transplantation or dental work.

In some embodiments, the dental work includes dental cleaning.

In some embodiments, the subject has thrombocytopenia.

In some embodiments, the subject further is in need of treatment for cancer, liver disease, vitamin B12 deficiency, a systemic viral infection, a systemic bacterial infection, sepsis, dengue fever or an immune disorder.

In some embodiments, the liver disease is chronic viral hepatitis, nonalcoholic steatohepatitis, alcoholic liver disease, liver failure or sepsis.

In some embodiments, the thrombocytopenia is chronic immune (idiopathic) thrombocytopenic purpura, radiation-induced thrombocytopenia, chemotherapy-induced thrombocytopenia, HIV/AIDS-induced thrombocytopenia, anemia-induced thrombocytopenia, thrombotic thrombocytopenic purpura or neonatal alloimmune thrombocytopenia.

In some embodiments, the subject is likely to develop thrombocytopenia due to the administration of chemotherapy or radiotherapy and 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid is administered prophylactically such that the subject is at decreased risk for bleeding.

In some embodiments, the subject has a durable platelet response.

In some embodiments, the subject has a transient platelet response.

In some embodiments, the subject maintains a platelet response.

In some embodiments, the subject has had less than 3 lines of prior therapy.

In some embodiments, the subject has had 3 or more lines of prior therapy.

In some embodiments, the subject has a history of splenectomy.

In some embodiments, the subject has no history of splenectomy.

In some embodiments, the subject concomitantly uses steroid medications.

In some embodiments, the steroid is prednisone.

In some embodiments, the subject reduces steroid use upon administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid.

In some embodiments, the subject discontinues steroid use upon administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid. In some embodiments, the subject permanently discontinues steroid use upon administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid.

In some embodiments, the platelet response is maintained for about 1 week, about 2 weeks, about 3 weeks or about 4 weeks after discontinuation of administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid. In some embodiments, the platelet count is maintained indefinitely.

### BRIEF DESCRIPTIONS OF THE DRAWINGS

FIG. 1 includes a graph illustrating the time-course of platelet count increases above baseline over the 28 day study period upon administration to the study subject a placebo or E5501 in the amounts of 2.5 mg, 5.0 mg, 10 mg and 20 mg.

FIG. 2 includes a graph illustrating the median platelet count (K/mm³) from baseline for the Full Analysis Set (FAS) population over 18 weeks.

FIG. 3 includes a graph illustrating the median platelet count (K/mm³) from baseline for the Sufficient Exposure population over 18 weeks.

FIG. 4 includes a chart illustrating the time distribution from the date of the last dose of E5501 in the rollover study versus the incidence of recurrence of thrombocytopenia.

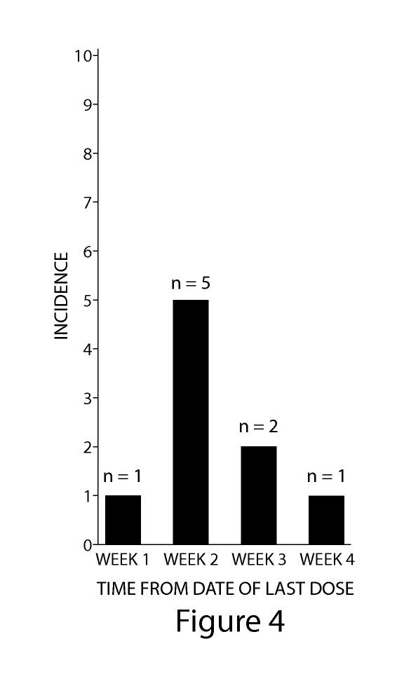

FIG. 5 includes a Kaplan Meier graph illustrating the time to the first bleeding event in the safety population.

### DETAILED DESCRIPTION OF THE INVENTION

### Definitions

In order to more clearly and concisely describe the subject matter of the claims, the following definitions are intended to provide guidance as to the meaning of terms used herein.

As used herein, the articles “a” and “an” mean “one or more” or “at least one,” unless otherwise indicated. That is, reference to any element of the present invention by the indefinite article “a” or “an” does not exclude the possibility that more than one of the element is present.

As used herein, the language “platelet response” refers to the change in platelet count that occurs in a subject upon administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid. The language “rapidly increasing the platelet response” refers to the stimulation of the platelet count in a subject after administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid. In some embodiments, the rapid increase in the platelet response includes the achievement of a platelet count of about ≧50,000/mm³ in a subject not receiving steroid medications with an initial platelet count of about <30,000/mm³ in less than about 14 days. In some embodiments, the rapid increase in the platelet response includes the achievement of a platelet count of about ≧50,000/mm³ in a subject not receiving steroid medications with an initial platelet increase in the platelet response includes the achievement of a platelet count of about ≧20,000/mm³ above the initial platelet count in a subject receiving steroids with an initial platelet count of about ≧30,000/mm³ but about <50,000/mm³ in less than about 14 days. In some embodiments, the rapid increase in the platelet response includes the achievement of a platelet count of about ≧20,000/mm³ above the initial platelet count in a subject receiving steroids with an initial platelet count of about ≧30,000/mm³ but about <50,000/mm³ in less than about 7 days. In some embodiments, rapid increase of the platelet response includes the achievement of a platelet count of at least about 50,000/mm³ in less than about 14 days. In some embodiments, rapid increase of the platelet response includes the achievement of a platelet count of at least about 50,000/mm³ in less than about 7 days.

In some embodiments, the platelet response is increased by at least about 5%, by about 10%, by about 15%, by about 20%, by about 25%, by about 30%, by about 35%, by about 40%, by about 45%, by about 50%, by about 55%, by about 60%, by about 65%, by about 70%, by about 75%, by about 80%, by about 85%, by about 90%, by about 95% or by about 100% over the initial platelet count. In some embodiments, the platelet response is increased by between at least about 10,000/mm³ and about 400,000/mm³. In some embodiments, the net change in the platelet response is between at least about 10,000/mm³ and about 400,000/mm³.

As used herein, the term “subject” refers to animals such as mammals, including, but not limited to, humans, primates, cows, sheep, goats, horses, pigs, dogs, cats, rabbits, guinea pigs, rats, mice or other bovine, ovine, equine, canine, feline, rodent or murine species. In some embodiments, the subject is a human. In some embodiments, the subject is at risk for bleeding due at least in part to a low platelet count. In some embodiments, the subject is in need of treatment that may induce bleeding. In some embodiments, the subject has active bleeding (e.g., central nervous system, gastrointestinal or genitourinary bleeding).

In some embodiments, the subject has thrombocytopenia. As used herein, the term “thrombocytopenia” refers to the abnormally low number of platelets in the blood.

In some embodiments, thrombocytopenia includes chronic immune (idiopathic) thrombocytopenic purpura, radiation-induced thrombocytopenia, chemotherapy-induced thrombocytopenia, HIV/AIDS-induced thrombocytopenia, anemia-induced thrombocytopenia, thrombotic thrombocytopenic purpura or neonatal alloimmune thrombocytopenia.

In some embodiments, the subject is using steroid medications. The language “steroid medications” refers to those steroid therapeutic agents that are administered concomitantly to the subject with 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid. Examples of steroid medications include, for example, corticosteroids (e.g., glucocorticoids and/or mineralocorticoids) such as methylprednisone, prednisone, corticosterone, cortisone, aldosterone, methylprednisolone, dexamethasone, hydrocortisone and combinations thereof.

In some embodiments, the subject has a history of splenectomy (e.g., a surgical procedure used to partially or completely remove the spleen). In some embodiments, the subject has no history of splenectomy.

In some embodiments, the subject has had less than three lines of prior therapy to increase platelet response or otherwise treat ITP. The language “lines of prior therapy” include any therapy, for example, surgical procedures or administration of therapeutic agents other than 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid, in order to stimulate platelet response. In some embodiments, the subject has had three or more lines of prior therapy. Examples of prior lines of therapy include, for example, splenectomy, platelet transfusions, plasmapheresis, hematopoietic stem cell transplantation and/or administration of therapeutic agents, including steroid medications (e.g., prednisone, dexamethasone, methylprednisone), intravenous anti-D, IVIg, antifibrinolytics (e.g., tranexamic acid, epsilon-aminocaproic acid) romiplostim, eltrombopag, mycophenolate mofetil, azathioerine, cyclosporin A, cyclophosphamide, danazol, dapsone, rituximab, campath-H, vinca alkaloids (e.g., vincristine) and combinations thereof. In some embodiments, the line of therapy is the administration of a steroid medication. In some embodiments, the line of therapy is a splenectomy.

As used herein, the term “treatment that may induce bleeding” refers to treatment with a risk of causing bleeding in a subject having thrombocytopenia or a low platelet count. In some embodiments, the treatment includes surgery, the administration of a therapeutic agent, radiation therapy or a combination thereof. The term “surgery,” as used herein, refers to a medical procedure that may involve the removal of diseased tissue, repair of damaged tissue, a diagnostic procedure or the examination of tissue to determine that type of disease is present. In some embodiments, surgery refers to a diagnostic procedure (e.g., a biopsy, endoscopy, colonoscopy, bone marrow aspiration, bronchoscopy, cardiac catheterization, colposcopy, hysteroscopy, joint aspiration, laparoscopy, mediastinoscopy, ophthalmoscopy, sigmoidoscopy, spinal tap, thoracentesis, thorascopy), the administration of an anesthetic, administration of an epidural, a transplantation or dental a procedure (e.g., dental cleaning, root canal, wisdom teeth extraction, dental implants, fillings, orthodontics/braces).

In some embodiments, the administration of a therapeutic agent includes the administration of valproic acid, methotrexate, carboplatin, interferon, isotretinoin, H2 blockers, proton pump inhibitors or heparin or the administration of chemotherapy. As used herein, the term “chemotherapy” refers to the treatment of a disease by chemicals that kills cells, including microorganisms and cancer cells. Examples of chemotherapies include, for example, cisplatin, carboplatin, oxaplatin, mechlorethamine, cyclophosphamide, chlorambucil, ifosfamide, azathioprme, mercaptopurine, vincristine, vinblastin, vinorelbine, vindesine, paclitaxel, docetael, etoposide, teniposide, irinotecan, topotecan, amsacrine, dactinomycin, doxorubicin, epirubin and bleomycin. As used herein, the term “radiation therapy” refers to the medical use of ionizing radiation as part of treatment, e.g., cancer treatment to control malignant cells.

In some embodiments, the subject is likely to develop thrombocytopenia due to chemotherapy or radiotherapy and 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid is administered prophylactically such that the subject is at decreased risk for bleeding.

In some embodiments, the subject has had a low platelet count within at least about 1 month of treatment, within at least about 14 days of treatment, within at least about 7 days of treatment or at least about the time of treatment.

As used herein, the term “bleeding” refers to any bleeding, as measured by the World Health Organization's Bleeding Scale (e.g., Grade 0=no bleeding; Grade 1=petechial bleeding; Grade 2=clinical significant mild blood loss; Grade 4=gross (severe) blood loss, requiring transfusion; Grade 5=debilitating blood loss, retinal or cerebral associated with fatality). In some embodiments, the bleeding may be due to a low platelet count.

The term “low platelet count” refers to a platelet count lower than the normal physiological platelet count of a healthy subject, e.g., between about 150,000/mm³ and about 450,000/mm³. In one embodiment, the low platelet count is less than about 150,000/mm³, less than about 100,000/mm³, less than about 80,000/mm³, less than about 50,000/mm³ or less than about 30,000/mm³. One of skill in the art would appreciate that the platelet count may vary depending on the technique (e.g., manual counting or machine counting) and/or laboratory utilized to count platelets.

As used herein, the term “initial platelet count” refers to the platelet count at the time prior to administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid. In some embodiments, the subject not receiving steroid medications has an initial platelet count of less than or about equal to 10,000/m³, 20,000/mm³, or 30,000/mm³ and the platelet response is increased in less than about 7 days to greater than or equal to about 50,0000/mm³. In some embodiments, the subject receiving steroid medications as an initial platelet count of between about 10,000/m³, about 20,000/mm³, or about 30,000/mm³ and about 50,000/mm³.

In some embodiments, the low platelet count is caused by vitamin B12 or folic acid deficiency, cancer (e.g., leukemia), myelodysplastic syndrome, an immune disorder, liver disease (e.g., chronic viral hepatitis, nonalcoholic steatohepatitis, alcoholic liver disease or liver failure), sepsis, a systemic viral or bacterial infection, dengue fever, congenital amegokaryocytic thrombocytopenia, thrombocytopenia absent radius syndrome, fanconi anemia, Bernard-Soulier syndrome, May Hegglin anomaly, grey platelet syndrome, Alport syndrome, immune (idiopathic) thrombocytopenic purpura (ITP), thrombotic thromboycytopenic purpura (TTP), hemolytic-uremic syndrome, disseminated intravascular coagulation, paroxysmal nocturnal hemoglobinuria, antiphospholipid syndrome, systemic lupus erythematosus, post transfusion purpura, neonatal alloimmune thrombocytopenia, splenic sequestration of platelets due to hypersplenism or HIV-associated thrombocytopenia.

The term “1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid,” as used herein, refers to:

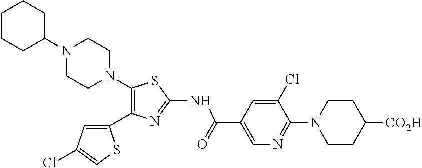

and its pharmaceutically acceptable salts, including the maleic acid salt. It is also referred to as E5501.

U.S. Patent Application Publication No. 20050153977, the entire contents of which are incorporated herein in their entirety, discloses methods of synthesizing 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid.

As used herein, the term “effective amount” refers to the amount of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid necessary to achieve a desired effect. The term “desired effect” refers generally to any result that is anticipated by the skilled artisan when 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid is administered to a subject. In some embodiments, the desired effect is the rapid increase in platelet, response. In some embodiments, the desired effect is a reduction of the risk of bleeding. In some embodiments, the desired effect is the complete elimination of the risk of bleeding. The exact amount required will vary from subject to subject, depending on the species, age, and general condition of the subject, the severity of the diseases, its mode of administration, and the like. In some embodiments, the effective dose is between about 1 mg and about 100 mg per day, between about 5 mg and about 50 mg per day, or between about 10 mg and about 40 mg per day. In some embodiments, the effective dose is about 5 mg, about 10 mg, about 15 mg, about 20 mg, about 25 mg, about 30 mg, about 35 mg, about 40 mg, about 45 mg, about 50 mg, about 55 mg, about 60, about 65 mg, about 70 mg, about 75 mg, about 80 mg, about 85 mg, about 90 mg, about 95 mg or about 100 mg per day.

In some embodiments, the effective amount is an effective periodic dose. As used herein, the term “effective periodic dose” refers to the amount effective to achieve the increase in the platelet response in a subject. In some embodiments, the effective periodic dose is a once daily dose, a twice daily dose, a thrice daily dose, a dose administered every other day, a weekly dose or a monthly dose. In some embodiments, the effective periodic dose is administered for about 1 month, for about 2 months, for about 3 months, for about 4 months, for about 5 months, for about 6 months or for about 7 months. The exact amount required will vary from subject to subject, depending on the species, age, and general condition of the subject, the severity of the diseases, its mode of administration, and the like.

In some embodiments, the subject receives a dosage adjustment of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid in the form of an upward or downward titration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid. The language “upward titration” refers to the administration of an increased dosage of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid from a specific dose until a desired platelet response is achieved. In some embodiments, the upward titration is achieved by increasing the amount of the dose and/or the frequency of the dose. The language “downward titration” refers to the administration of a lower dosage of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid from a specific dose until a desired platelet response is achieved. In some embodiments, the downward titration is achieved by decreasing the amount of the dose and/or the frequency of the dose.

As used herein, the term “decreased risk for bleeding” refers to any reduction in the risk of bleeding as measured by the WHO's Bleeding Scale. In some embodiments, the decrease in risk is at least about a 10% decrease, about a 20% decrease, about a 30% decrease, about a 40% decrease, about a 50% decrease, about a 60% decrease, about a 70% decrease, about an 80% decrease, about a 90% decrease or the elimination of the risk for bleeding.

As used herein, the term “detecting the platelet response” refers to any technique used in the art to measure or detect the platelet response. One of ordinary skill in the art would be able to determine the appropriate technique for measuring or detecting the platelet response with no more than routine experimentation.

As used herein, the term “response rate” refers to the increase in the platelet response (e.g., a platelet count of about 50,000/m³) over time after administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid. In some embodiments, the response rate results in a platelet count of greater than or equal to about 50,000/mm³ after about 7 days, about 14 days, about 21 days or about one month of administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid. In still other embodiments, the response rate is an achievement of any weekly platelet count of greater than or equal to about 50,000/mm³ for any four weeks during administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid. In some embodiments, the subject has a response rate of at least about 10%, about 25%, about 50% or about 80% over initial platelet levels at about 28 days compared to a subject receiving a placebo. In some embodiments, the subject has a response rate of at least about 5%, about 10%, about 25%, about 50%, about 70%, about 90% or about 98% over initial platelet levels at about 7 days compared to a subject receiving a placebo.

As used herein, the term “sustained platelet response” refers to a platelet response (e.g., a platelet count of about 50,000/mm³) that is maintained for at least about 7 days, about 14 days about 21 days, about 28 days, about 2 months, about 3 months, about 4 months, about 5 months, about 6 months or about 7 months. In some embodiments, the subject has a sustained platelet response of at least about 25%, about 30%, about 40%, about 50%, about 75% or about 77% compared to a subject receiving a placebo.

The language “durable platelet response,” as used herein refers to a platelet response rate of at least about 75% in about 14 weeks of a 24-week treatment period in which no rescue medications are required to increase the platelet response. The language “rescue medication” refers to therapeutic agents administered to a subject in addition to 1-(3-chloro-5-{[4-(4-chlorothiophen-2-yl)-5-(4-cyclohexylpiperazin-1-yl) thiazol-2-yl]carbamoyl}pyridine-2-yl)piperidine-4-carboxylic acid in order to stimulate platelet production.

The language “transient platelet response,” refers to an achievement of a platelet response rate for any 4 consecutive weeks during a 24-week treatment period in the absence of a durable platelet response and in the absence of any rescue medication.

The language “overall platelet response” refers to the combined durable platelet response plus the transient platelet response.

The language “maintenance of platelet response,” as used herein, refers to the achievement of a durable platelet response over a 7 month period without an upward titration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid.

In some embodiments, the platelet response is maintained indefinitely. The language “maintained indefinitely” includes ameliorating or curing the subject from acute or chronic low platelet count, such that the subject no longer needs medication to treat a low platelet count.

The language “reduction of steroid use,” as used herein, refers to any decrease in the amount of concomitant steroid intake as a result of a platelet response upon administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid. In some embodiments, the steroid use is reduced by about 5%, by about 10%, by about 15%, by about 20%, by about 25<by about 30%, by about 35%, by about 40%, by about 45%, by about 50%, by about 55%, by about 60%, by about 65%, by about 70%, by about 75%, by about 80%, by about 85%, by about 90%, by about 95% or by about 100%. In some embodiments, there is a permanent discontinuation of steroid use. In some embodiments, the reduction of steroid use or permanent discontinuation of steroid use occurs immediately upon administration of -(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid, or within about 7 days, about 14 days, about 21 days, about 28 days, about 1 month, about 2 months, about 3 months, about 4 months, about 5 months, about 6 months or about 7 months of treatment with -(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid.

It will be appreciated that according to the methods of the present invention, 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid may be administered using any amount and any route of administration effective for increasing the platelet response. It will be understood, however, that the administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid will be decided by the attending physician within the scope of sound medical judgment. In some embodiments, 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid may administered orally, rectally, intravenously, intraperitoneally, intramuscularly, intraarterially, intradermally, subcutaneously, transdermally intratracheally, subcutaneously, by inhalation, nasally, naval, by suppository or by direct injection into a tissue, or absorbed by topical or mucosal administration.

### EXEMPLIFICATION

The methods of this invention can be understood further by the following examples. It will be appreciated, however, that these examples do not limit the invention. Variations of the invention, now known or further developed, are considered to fall within the scope of the present invention as described herein and as hereinafter claimed.

### Example 1

### A Phase 2, Double Blind, Randomized, Dose-Ranging, Placebo-Controlled, Parallel Group Study of E5501 Tablets Taken Orally Once Daily for 28 Days in Subjects with Chronic Idiopathic Thrombocytopenia Purpura (ITP)

### Introduction

This was a phase 2, multi-center, double-blind, randomized, placebo-controlled, dose-ranging, parallel group study of E5501 tablets, used in the treatment of subjects with chronic idiopathic thrombocytopenic purpura (ITP) refractory to or relapsed after at least one prior ITP therapy. Subjects who met all entry criteria were randomly assigned in a 3:3:3:3:1 ratio to one of the following five treatment groups dosed daily for 28 days: 1) E5501 2.5 mg; 2) E5501 5 mg; 3) E5501 10 mg; 4) E5501 20 mg; or 5) placebo (PBO). Each E5501 dosing group consisted of 15 subjects while the PBO group consisted of 5 subjects. All study subjects were evaluated weekly (days 7, 14, 21 and 28) for safety and efficacy while receiving study treatment, with a final assessment for safety and effectiveness performed 4 weeks after the last study dose (day 56) unless the subject entered the follow-on study. Platelet counts were also measured on day 3 and day 5.

The primary objective of this study was to assess platelet count response to E5501 on day 28. The response rate was defined as the proportion of subjects who were not on steroids with a screening visit B platelet count <30,000/mm³ and who achieved a platelet count ≧50,000/mm³, together with the proportion of subjects receiving steroids with a screening visit B platelet count ≧30,000/mm³ but <50,000/mm³ who achieved a platelet count ≧20,000/mm³ above their screening visit B platelet count. Secondary objectives of the study included the evaluation of other markers of effectiveness, including changes in peripheral blood platelet count, the proportion of subjects who achieved a platelet count ≧50,000/mm³ and ≧100,000/mm³, and the proportion of subjects who achieved a doubling of their platelet count at screening visit B and the assessment of the safety and tolerability of E5501.

### Efficacy Endpoints and Planned Analyses

Primary efficacy endpoint. The primary endpoint in this study was E5501 responder rate on day 28. A responder is defined as a subject with screening visit B platelet count <30,000/mm³ who achieves a platelet count ≧50,000/mm³ or a subject receiving steroids with a screening visit B platelet count ≧30,000/mm³ but <50,000/mm³ who achieves a platelet count ≧20,000/mm³ above their screening visit B platelet count.

### Secondary Endpoints

The secondary endpoints included changes from screening visit B in peripheral blood platelet counts over time; the proportion of subjects who achieved a platelet count of ≧50,000/mm³ or ≧100,000/mm³ on day 28 and the proportion of subjects whose platelet count doubled compared to their platelet count at Screening Visit B.

The safety and tolerability parameters included adverse events, clinical laboratory parameters, study drug exposure, vital electrocardiograms, concomitant medications, serum pregnancy tests for women of childbearing potential and physical examinations including height, weight, general appearance and recording of vital signs.

### Analysis Populations

The populations of interest were defined as follows:
The randomized population included all subjects who were randomized into the study.
The safety population included all randomized subjects who receive at least one dose of study drug and have at least one safety assessment.
The pharmacokinetic (PK) population included all subjects who provided blood sample for analysis of E5501. A subject was included in this population if the subject had at least one blood draw for PK assessment.
The full analysis set (FAS) included all subjects who provided adequate data to derive at least one efficacy assessment. Subjects were included in this population if the subject had at least one post-baseline platelet count assessment.
The per protocol (PP) population included the subset of subjects who were in both the safety and full analysis population, and did not meet any of the following criteria:
   A subject's steroids dosage was increased during study;
   A subject used a prohibited medication that affected the assessment of study endpoint;
   A subject skipped more than 15% of daily doses for reason other than high platelet count, or was on medication for less than 25 days and did not continue into the extension study;
   A subject did not have a day 28 platelet count within the visit windoe. The day 28 visit window was defined to be from day 25, to last dose day+1, inclusively;
   Other major protocol violations that impacted the platelet endpoint evaluation.
      Results from Planned Analyses

Subject Disposition. Sixty four subjects were randomly assigned to treatment in this study. All treated subjects provided adequate efficacy data and therefore were included in the FAS population. The number and percentage of subjects who completed treatment or discontinued prematurely were summarized by treatment groups as shown in Table 1. In addition, the number and percentage of subjects who withdrew were summarized by reason for discontinuation by treatment group. The majority (>85%) of subjects completed the study across all dose groups for the FAS, randomized, and safety populations. Seven subjects who discontinued the study early all were from E5501 dose groups: two subjects in each of 2.5, 10 and 20 mg dose groups and one subject in the 5 mg dose group. Of these seven subjects, two subjects (one for each of the 5 and 10 mg E5501 dose groups) withdrew due to an AE (musculoskeletal chest pain (5 mg); myocardial infarction, transient ischemic attack, pneumonia and retinal artery occlusion (10 mg)) and 2 subjects (from 20 mg) withdrew due to excessively increased platelet counts (≧500,000/mm³).

TABLE 1

| | | E5501 | E5501 | E5501 | E5501 | Total |
|---|---|---|---|---|---|---|
| Population | Placebo | 2.5 mg | 5 mg | 10 mg | 20 mg | E5501 |
| Randomized, N^{a} | 5 | 15 | 15 | 14 | 15 | 59 |
| Safety Population, N^{a} | 5 | 15 | 15 | 14 | 15 | 59 |
| Full Analysis Set, N^{a} | 5 | 15 | 15 | 14 | 15 | 59 |
| Completed Study, n (%) | 5 (100.0) | 13 (86.7) | 14 (93.3) | 12 (85.7) | 13 (86.7) | 52 (88.1) |
| Discontinued Study, n (%) | 0 | 2 (13.3) | 1 (6.7) | 2 (14.3) | 2 (13.3) | 7 (11.9) |
| Primary Reason for Discontinuation | | | | | | |
| Adverse Event or SAE | 0 | 0 | 1 (6.7) | 1 (7.1) | 0 | 2 (3.4) |
| Platelet Count Increase to ≧500 K/mm³ | 0 | 0 | 0 | 0 | 2 (13.3) | 2 (3.4) |
| Withdrew Consent | 0 | 1 (6.7) | 0 | 1 (7.1) | 0 | 2 (3.4) |
| Other | 0 | 1 (6.7) | 0 | 0 | 0 | 1 (1.7) |
| Per Protocol Population, N | 5 | 13 | 13 | 12 | 12 | 50 |
| Completed Study, n (%) | 5 (100.0) | 13 (100.0) | 13 (100.0) | 12 (100.0) | 12 (100.0) | 50 (100.0) |
| Discontinued Study, n (%) | 0 | 0 | 0 | 0 | 0 | 0 |
| Pharmacokinetic Population, N | 5 | 14 | 15 | 14 | 15 | 58 |
| Completed Study, n (%) | 5 (100.0) | 13 (92.9) | 14 (93.3) | 12 (85.7) | 13 (86.7) | 52 (89.7) |
| Discontinued Study, n (%) | 0 | 1 (7.1) | 1 (6.7) | 2 (14.3) | 2 (13.3) | 6 (10.3) |
| Primary Reason for Discontinuation | | | | | | |
| Adverse Event or SAE | 0 | 0 | 1 (6.7) | 1 (7.1) | 0 | 2 (3.4) |
| Platelet Count Increase to ≧500 K/mm³ | 0 | 0 | 0 | 0 | 2 (13.3) | 2 (3.4) |
| Withdrew Consent | 0 | 0 | 0 | 1 (7.1) | 0 | 1 (1.7) |
| Other | 0 | 1 (7.1) | 0 | 0 | 0 | 1 (1.7) |

^{a}The randomized, safety and full analyses populations were identical.

### Subject Demographics and Baseline Characteristics

Subject demographics were summarized by treatment group, as shown in Table 2. Continuous variables such as age, weight and height at screening, baseline platelet count category (≦15,000/mm³ versus >15,000/mm³), history of splenectomy, number of lines of prior therapy and prior steroid use are summarized by descriptive statistics (m, mean, standard deviation, median, minimum and maximum). Categorical variables such as sex, race and reproductive status were summarized by number and percentage.

The analyses of demographic and baseline characteristics was performed for all five populations. In general, subject demographics and baseline characteristics are well balanced across the placebo and all four E5501 dose groups.

TABLE 2

| | | E5501 | E5501 | E5501 | E5501 | Total |
|---|---|---|---|---|---|---|
| | Placebo | 2.5 mg | 5 mg | 10 mg | 20 mg | E5501 |
| Category | (N = 5) | (N = 15) | (N = 15) | (N = 14) | (N = 15) | (N = 59) |
| Age (Year) | | | | | | |
| n | 5 | 15 | 15 | 14 | 15 | 59 |
| Mean (SD) | 39.6 (20.63) | 52.9 (17.84) | 55.6 (18.03) | 57.5 (17.88) | 47.9 (16.54) | 53.4 (17.50) |
| Median | 29.0 | 56.0 | 58.0 | 60.0 | 44.0 | 57.0 |
| Min, Max | 23, 73 | 20, 81 | 18, 79 | 22, 82 | 22, 77 | 18, 82 |
| Sex, n (%) | | | | | | |
| Male | 2 (40.0) | 6 (40.0) | 6 (40.0) | 6 (42.9) | 4 (26.7) | 22 (37.3) |
| Female | 3 (60.0) | 9 (60.0) | 9 (60.0) | 8 (57.1) | 11 (73.3) | 37 (62.7) |
| Race, n (%) | | | | | | |
| Black, African Heritage | 0 | 2 (13.3) | 0 | 1 (7.1) | 2 (13.3) | 5 (8.5) |
| Asian | 0 | 1 (6.7) | 2 (13.3) | 0 | 1 (6.7) | 4 (6.8) |
| Pacific Islander | 0 | 0 | 0 | 0 | 0 | 0 |
| Hispanic | 1 (20.0) | 1 (6.7) | 1 (6.7) | 1 (7.1) | 1 (6.7) | 4 (6.8) |
| American Indian | 0 | 0 | 0 | 0 | 0 | 0 |
| White | 4 (80.0) | 11 (73.3) | 11 (73.3) | 12 (85.7) | 10 (66.7) | 44 (74.6) |
| Other | 0 | 0 | 1 (6.7) | 0 | 1 (6.7) | 2 (3.4) |
| Childbearing Potential, n (%) | | | | | | |
| Yes | 2 (66.7) | 3 (33.3) | 3 (33.3) | 2 (25.0) | 7 (63.6) | 15 (40.5) |
| Sterile | 0 | 2 (22.2) | 0 | 1 (12.5) | 1 (9.1) | 4 (10.8) |
| Postmenopausal | 1 (33.3) | 4 (44.4) | 6 (66.7) | 5 (62.5) | 3 (27.3) | 18 (48.6) |
| Height (cm) | | | | | | |
| n | 5 | 12 | 14 | 13 | 13 | 52 |
| Mean (SD) | 162.8 (13.59) | 168.8 (9.02) | 169.9 (13.33) | 166.8 (10.16) | 170.0 (11.92) | 168.9 (11.06) |
| Median | 155.0 | 171.5 | 169.0 | 165.0 | 165.0 | 168.0 |
| Min, Max | 152, 180 | 152, 178 | 152, 193 | 150, 185 | 155, 196 | 150, 196 |
| Weight (kg) | | | | | | |
| n | 5 | 14 | 15 | 14 | 14 | 57 |
| Mean (SD) | 72.6 (8.50) | 80.0 (16.31) | 79.9 (17.23) | 76.3 (14.05) | 86.4 (21.27) | 80.6 (17.33) |
| Median | 74.0 | 81.0 | 77.0 | 74.5 | 84.0 | 77.0 |
| Min, Max | 63, 83 | 53, 104 | 56, 109 | 57, 102 | 59, 124 | 53, 124 |
| Prior History of Splenectomy | | | | | | |
| Yes | 2 (40.0) | 5 (33.3) | 4 (26.7) | 3 (21.4) | 6 (40.0) | 18 (30.5) |
| No | 3 (60.0) | 10 (66.7) | 11 (73.3) | 11 (78.6) | 9 (60.0) | 41 (69.5) |
| Prior Steroids Usage | | | | | | |
| Yes | 3 (60.0) | 4 (26.7) | 8 (53.3) | 6 (42.9) | 10 (66.7) | 28 (47.5) |
| No | 2 (40.0) | 11 (73.3) | 7 (46.7) | 8 (57.1) | 5 (33.3) | 31 (52.5) |
| Baseline Platelet Count | | | | | | |
| Category | | | | | | |
| <=15K/mm3 | 2 (40.0) | 5 (33.3) | 2 (13.3) | 5 (35.7) | 4 (26.7) | 16 (27.1) |
| >15K/mm3 | 3 (60.0) | 10 (66.7) | 13 (86.7) | 9 (64.3) | 11 (73.3) | 43 (72.9) |
| Number of Lines of Prior | | | | | | |
| Therapies | | | | | | |
| 1 | 0 | 0 | 5 (33.3) | 1 (7.1) | 3 (20.0) | 9 (15.3) |
| 2 | 1 (20.0) | 3 (20.0) | 4 (26.7) | 3 (21.4) | 4 (26.7) | 14 (23.7) |
| 3 | 1 (20.0) | 4 (26.7) | 1 (6.7) | 5 (35.7) | 3 (20.0) | 13 (22.0) |
| 4 | 2 (40.0) | 3 (20.0) | 0 | 1 (7.1) | 2 (13.3) | 6 (10.2) |
| 5 | 1 (20.0) | 3 (20.0) | 1 (6.7) | 1 (7.1) | 3 (20.0) | 8 (13.6) |
| 6 | 0 | 1 (6.7) | 2 (13.3) | 1 (7.1) | 0 | 4 (6.8) |
| 7 | 0 | 0 | 2 (13.3) | 1 (7.1) | 0 | 3 (5.1) |
| 8 | 0 | 0 | 0 | 1 (7.1) | 0 | 1 (1.7) |
| 9 | 0 | 1 (6.7) | 0 | 0 | 0 | 1 (1.7) |

Demographics and baseline characteristics for the FAS were identical to those for the randomized and safety populations.

### Efficacy Results

Primary Efficacy Endpoint. The analysis of efficacy was performed both on the FAS population and the PP population at day 28 visit with LOCF and observed case (OC) approaches. The primary analysis population was the FAS. Fisher's exact test was used to examine the difference of response rates among each treatment pair. All p-values are nominal without adjustment for multiplicity.

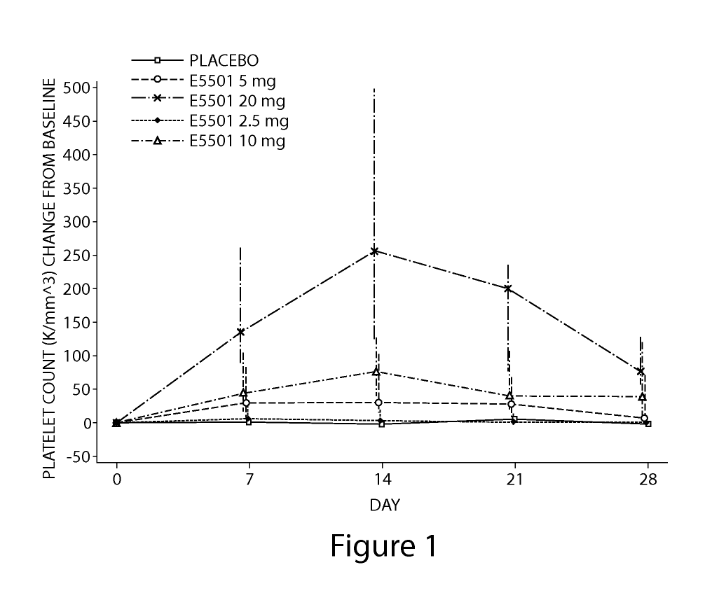

For the FAS, the primary endpoint (a day 28 responder rate significantly higher than that of placebo) was achieved for the 20 mg group (p=0.0036; Table 3). As shown in FIG. 1, E5501 markedly increased platelet counts from baseline. Increased platelet counts that met the criteria for positive response at day 28 were observed for 0%, 13.3%, 53.3%, 50% and 80% of subjects in the placebo, 2.5 mg, 5 mg, 10 mg and 20 mg E5501, respectively. A significant difference was also obtained between 20 mg and 2.5 mg groups (p=0.0007).

TABLE 3

| | | | | | | | Trend |
|---|---|---|---|---|---|---|---|
| | | E5501 | E5501 | E5501 | E5501 | Total | Test |
| Category | Placebo | 2.5 mg | 5 mg | 10 mg | 20 mg | E5501 | P-value |
| Full Analysis Population | | | | | | | |
| Responder, n (%) | | | | | | | |
| Yes | 0 | 2 (13.3) | 8 (53.3) | 7 (50.0) | 12 (80.0) | 29 (49.2) | |
| No | 5 (100.0) | 13 (86.7) | 7 (46.7) | 7 (50.0) | 3 (20.0) | 30 (50.8) | |
| Total | 5 | 15 | 15 | 14 | 15 | 59 | |
| P-Values^{a} | | | | | | | |
| vs. Placebo | | 1.0000 | 0.0547 | 0.1060 | 0.0036 | | |
| vs. E5501 2.5 mg | | | 0.0502 | 0.0502 | 0.0007 | | |
| vs. E5501 5 mg | | | | 1.0000 | 0.2451 | | |
| vs. E5501 10 mg | | | | | 0.1281 | | |
| Exact Cochran-Armitage | | | | | | | 0.000123 |
| trend test | | | | | | | |
| Exact logistic trend test | | | | | | | 0.000100 |
| Per Protocol Population | | | | | | | |
| Responder, n (%) | | | | | | | |
| Yes | 0 | 2 (15.4) | 6 (46.2) | 7 (58.3) | 9 (75.0) | 24 (48.0) | |
| No | 5 (100.0) | 11 (84.6) | 7 (53.8) | 5 (41.7) | 3 (25.0) | 26 (52.0) | |
| Total | 5 | 13 | 13 | 12 | 12 | 50 | |
| P-Values^{a} | | | | | | | |
| vs. Placebo | | 1.0000 | 0.1141 | 0.0441 | 0.0090 | | |
| vs. E5501 2.5 mg | | | 0.2016 | 0.0414 | 0.0048 | | |
| vs. E5501 5 mg | | | | 0.6951 | 0.2262 | | |
| vs. E5501 10 mg | | | | | 0.6668 | | |
| Exact Cochran-Amitage | | | | | | | 0.00061 |
| trend test | | | | | | | |
| Exact logistic trend test | | | | | | | 0.00060 |

^{a}P-values calculated using Fisher's Exact test.

Similarly, the differences in the PP population between 20 mg E5501 and placebo and between 20 mg and 2.5 mg were statistically significant.

Analysis of the primary efficacy endpoint based on the OC method for both the FAS and PP populations yields similar and consistent results.

In the subgroup analyses, the data indicate that the majority of subjects responded to 20 mg E5501 regardless of baseline platelet count, baseline use of concomitant ITP medication or prior splenectomy (Table 4).

Approximately 44% of subjects were enrolled at site 132. To explore the potential for large site effect, the responder rates for the FAS from subjects enrolled at site 132 versus the other sites pooled are listed in Table 4. Similar responses were observed at this site, except that the response to 5 mg E5501 was 80% compared with 40% of subjects from other sites pooled. The difference between site 132 and other site pooled based on the baseline characteristics evaluation could not explain this observation.

The Exact Cochran-Armitage Trend test and LOGISTIC modeling for an exact conditional scores test were used to examine the dose-response relation for the FAS and PP population. The results indicated the that there was a strong, statistically significant trend towards an increase in response rate with increasing E5501 doses from 0 (placebo) up to 20 mg. The findings based on the two statistical evaluations are consistent.

TABLE 4

| | Visit | | E5501 | E5501 | E5501 | E5501 | Total |
|---|---|---|---|---|---|---|---|
| Category | Category | Placebo | 2.5 mg | 5 mg | 10 mg | 20 mg | E5501 |
| Site 132 | Day 28 | | | | | | |
| | Yes | 0 | 1 (16.7) | 4 (80.0) | 4 (57.1) | 7 (77.8) | 16 (59.3) |
| | No | 1 (100.0) | 5 (83.3) | 1 (20.0) | 3 (42.9) | 2 (22.2) | 11 (40.7) |
| | Total | 1 | 6 | 5 | 7 | 9 | 27 |
| Other Site | Day 28 | | | | | | |
| Pooled | Yes | 0 | 1 (11.1) | 4 (40.0) | 3 (42.9) | 5 (83.3) | 13 (40.6) |
| | No | 4 (100.0)2 | 8 (88.9) | 6 (60.0)2 | 4 (57.1) | 1 (16.7) | 19 (59.4) |
| | Total | 4 | 9 | 10 | 7 | 6 | 32 |
| Baseline Platelet | Day 28 | | | | | | |
| Cnt. ≦15K/mm³ | Yes | 0 | 0 | 1 (50.0) | 2 (40.0) | 3 (75.0) | 6 (37.5) |
| | No | 2 (100.0) | 5 (100.0) | 1 (50.0) | 3 (60.0) | 1 (25.0) | 10 (62.5) |
| | Total | 2 | 5 | 2 | 5 | 4 | 16 |
| Baseline Platelet | Day 28 | | | | | | |
| Cnt. >15K/mm³ | Yes | 0 | 2 (20.0) | 7 (53.8) | 5 (55.6) | 9 (81.8) | 23 (53.5) |
| | No | 3 (100.0) | 8 (80.0) | 6 (46.2) | 4 (44.4) | 2 (18.2) | 20 (46.5) |
| | Total | 3 | 10 | 13 | 9 | 11 | 43 |
| No Previous | Day 28 | | | | | | |
| History of | Yes | 0 | 1 (10.0) | 6 (54.5) | 6 (54.5) | 8 (88.9) | 21 (51.2) |
| Splenectomy | No | 3 (100.0) | 9 (90.0) | 5 (45.5) | 5 (45.5) | 1 (11.1) | 20 (48.8) |
| | Total | 3 | 10 | 11 | 11 | 9 | 41 |
| Previous History | Day 28 | | | | | | |
| of Splenectomy | Yes | 0 | 1 (20.0) | 2 (50.0) | 1 (33.3) | 4 (66.7) | 8 (44.4) |
| | No | 2 (100.0) | 4 (80.0) | 2 (50.0) | 2 (66.7) | 2 (33.3) | 10 (55.6) |
| | Total | 2 | 5 | 4 | 3 | 6 | 18 |
| Number of Lines | Day 28 | | | | | | |
| of Prior | Yes | 0 | 0 | 5 (55.6) | 3 (75.0) | 6 (85.7) | 14 (60.9) |
| Therapy <3 | No | 1 (100.0) | 3 (100.0) | 4 (44.4) | 1 (25.0) | 1 (14.3) | 9 (39.1) |
| | Total | 1 | 3 | 9 | 4 | 7 | 23 |
| Number of Lines | Day 28 | | | | | | |
| of Prior | Yes | 0 | 2 (16.7) | 3 (50.0) | 4 (40.0) | 6 (75.0) | 15 (41.7) |
| Therapy ≧3 | No | 4 (100.0) | 10 (83.3) | 3 (50.0) | 6 (60.0) | 2 (25.0) | 21 (58.3) |
| | Total | 4 | 12 | 6 | 10 | 8 | 36 |

Secondary Efficacy Endpoint. Analyses for secondary efficacy endpoints were performed by treatment group and by analysis visit using both LOCF and OC methods. The analysis visit was specified in the statistical analysis plan using both the target window and the allowed window in the analysis. Counts and percentages of responders (using the same responder criteria) are presented by treatment group and by analysis visit using the LOCF method in Table 5 below.

TABLE 5

| Visit | | E5501 | E5501 | E5501 | E5501 | Total |
|---|---|---|---|---|---|---|
| Category | Placebo | 2.5 mg | 5 mg | 10 mg | 20 mg | E5501 |
| Full Analysis Set | | | | | | |
| Total | 5 | 15 | 15 | 14 | 15 | 59 |
| Day 7 | | | | | | |
| Responder, n (%) | 0 | 1 (6.7) | 10 (66.7) | 9 (64.3) | 14 (93.3) | 34 (57.6) |
| Nonresponder, n (%) | 5 (100.0) | 14 (93.3) | 5 (33.3) | 5 (35.7) | 1 (6.7) | 25 (42.4) |
| P-values^{a} | | | | | | |
| vs. Placebo | | 1.0000 | 0.0325 | 0.0325 | 0.0004 | |
| vs. E5501 2.5 mg | | | 0.0017 | 0.0017 | <0.0001 | |
| vs. E5501 5 mg | | | | 1.0000 | 0.1686 | |
| vs. E5501 10 mg | | | | | 0.0801 | |
| Day 14 | | | | | | |
| Responder, n (%) | 0 | 3 (20.0) | 9 (60.0) | 11 (78.6) | 14 (93.3) | 37 (62.7) |
| Nonresponder, n (%) | 5 (100.0) | 12 (80.0) | 6 (40.0) | 3 (21.4) | 1 (6.7) | 22 (37.3) |
| P-values^{a} | | | | | | |
| vs. Placebo | | 0.5395 | 0.0379 | 0.0048 | 0.0004 | |
| vs. E5501 2.5 mg | | | 0.0604 | 0.0028 | 0.0001 | |
| vs. E5501 5 mg | | | | 0.4270 | 0.0801 | |
| vs. E5501 10 mg | | | | | 0.3295 | |
| Day 21 | | | | | | |
| Responder, n (%) | 0 | 2 (13.3) | 9 (60.0) | 7 (50.0) | 13 (86.7) | 31 (52.5) |
| Nonresponder, n (%) | 5 (100.0) | 13 (86.7) | 6 (40.0) | 7 (50.0) | 2 (13.3) | 28 (47.5) |
| P-values^{a} | | | | | | |
| vs. Placebo | | 1.0000 | 0.0379 | 0.1060 | 0.0014 | |
| vs. E5501 2.5 mg | | | 0.0209 | 0.0502 | 0.0001 | |
| vs. E5501 5 mg | | | | 0.7152 | 0.2148 | |
| vs. E5501 10 mg | | | | | 0.0502 | |
| Day 28 | | | | | | |
| Responder, n (%) | 0 | 2 (13.3) | 8 (53.3) | 7 (50.0) | 12 (80.0) | 29 (49.2) |
| Nonresponder, n (%) | 5 (100.0) | 13 (86.7) | 7 (46.7) | 7 (50.0) | 3 (20.0) | 30 (50.8) |
| P-values^{a} | | | | | | |
| vs. Placebo | | 1.0000 | 0.0547 | 0.1060 | 0.0036 | |
| vs. E5501 2.5 mg | | | 0.0502 | 0.0502 | 0.0007 | |
| vs. E5501 5 mg | | | | 1.0000 | 0.2451 | |
| vs. E5501 10 mg | | | | | 0.1281 | |

^{a}P-values calculated using Fisher's Exact test.

E5501 increased platelet counts relatively quickly; the responder rates reached 6.7%, 66.7%, 64% and 93.3% by Day 7 following treatment with E5501 2.5 mg, 5 mg, 10 mg and 20 mg, respectively. Approximately 90% of subjects who received E5501 20 mg achieved an increase in platelet count by ≧50,000/mm³ by Day 7 (Table 5).

The data further showed that for those subjects who achieved a platelet response on Day 7, approximately 30% to 80% of subjects who received ≧5 mg maintained their response for an additional 21 days. None of the subjects who received placebo or E5501 2.5 mg for 28 days achieved a sustained platelet response (Table 6).

TABLE 6

| | | E5501 | E5501 | E5501 | E5501 | Total |
|---|---|---|---|---|---|---|
| | Placebo | 2.5 mg | 5 mg | 10 mg | 20 mg | E5501 |
| Category | (N = 5) | (N = 13) | (N = 14) | (N = 12) | (N = 13) | (N = 52) |
| Subjects Who Maintained | | | | | | |
| Responder Status, n (%)^{a} | | | | | | |
| Yes | 0 | 0 | 4 (28.6) | 5 (41.7) | 10 (76.9) | 19 (36.5) |
| No | 5 (100.0) | 13 (100.0) | 10 (71.4) | 7 (58.3) | 3 (23.1) | 33 (63.5) |
| Total | 5 | 13 | 14 | 12 | 13 | 52 |
| P-Values^{b} | | | | | | |
| vs. Placebo | | | 0.5304 | 0.2445 | 0.0065 | |
| vs. E5501 2.5 mg | | | 0.0978 | 0.0149 | 0.0001 | |
| vs. E5501 5 mg | | | | 0.6828 | 0.0213 | |
| vs. E5501 10 mg | | | | | 0.1107 | |

Note:
The completer population included subjects who completed treatment with study drug at the end of the observation period.
^{a}Percentages are based on the total number of subjects with nonmissing data in the relevant treatment group.
^{b}P-values are based on Fisher's Exact test for each treatment pair.

### Safety Analysis Results

Fifty four out of the 59 E5501-treated subjects (84.7%) experienced a treatment emergent adverse event (TEAE). For 35 (59.3%) of the E5501-treated subjects, the TEAE was deemed to either possibly or probably related to E5501 (Table 7). Most of these events were mild in severity, transient and completely resolved. Fatigue (20.3%), headache (20.3%) and epistaxis (15.3%) were the only AEs occurring in ≧10% of E5501-treated subjects (Table 8).

Overall, 3 subjects reported 6 serious TAEAs (2.5 mg; n=2; 10 mg: n=4). In the 10 mg dose group, the 4 serious TEAEs (MI, TIA, retinal artery occlusion (all considered possibly drug related) and pneumonia (non related) all occurred in 1 subject; this subject had a history of MI and TIA. In the 2.5 mg dose group, 1 subject was diagnosed with thrombocytopenia and 1 with hemorrhagic gastritis. Both cases were classified by the investigators as grade 4 and not related to study drug (Table 9).

There were 4 cases of TEAE leading to withdrawal of study treatment and 3 cases of TEAE leading to study treatment interruption (Table 7).

TABLE 7

| | | E5501 | E5501 | E5501 | E5501 | Total |
|---|---|---|---|---|---|---|
| | Placebo | 2.5 mg | 5 mg | 10 mg | 20 mg | 501 |
| | (N = 5) | (N = 15) | (N = 15) | (N = 14) | (N = 15) | (N = 59) |
| Category | n (%) | n (%) | n (%) | n (%) | n (%) | n (%) |
| TEAEs | 4 (80.0) | 12 (80.0) | 13 (86.7) | 11 (78.6) | 14 (93.3) | 50 (84.7) |
| Treatment- | 1 (20.0) | 6 (40.0) | 11 (73.3) | 8 (57.1) | 10 (66.7) | 35 (59.3) |
| Related TEAEs | | | | | | |
| Serious TEAEs | 0 | 2 (13.3) | 0 | 1 (7.1) | 0 | 3 (5.1) |
| Treatment- | 0 | 0 | 0 | 1 (7.1) | 0 | 1 (1.7) |
| Related Serious | | | | | | |
| TEAEs | | | | | | |
| Death | 0 | 0 | 0 | 0 | 0 | 0 |
| TEAE Leading | 0 | 0 | 1 (6.7) | 1 (7.1) | 2 (13.3) | 4 (6.8) |
| to Study | | | | | | |
| Treatment | | | | | | |
| Withdrawal | | | | | | |
| TEAE Leading | 0 | 0 | 1 (6.7) | 0 | 2 (13.3) | 3 (5.1) |
| to Study | | | | | | |
| Treatment Dose | | | | | | |
| Interruption | | | | | | |

TABLE 8

| | | E5501 | E5501 | E5501 | E5501 | Total |
|---|---|---|---|---|---|---|
| | Placebo | 2.5 mg | 5 mg | 10 mg | 20 mg | E5501 |
| MedDRA | (N = 5) | (N = 15) | (N = 15) | (N = 14) | (N = 15) | (N = 59) |
| Preferred Term | n (%) | n (%) | n (%) | n (%) | n (%) | n (%) |
| Fatigue | 1 (20.0) | 2 (13.3) | 4 (26.7) | 4 (28.6) | 2 (13.3) | 12 (20.3) |
| Headache | 1 (20.0) | 4 (26.7) | 3 (20.0) | 1 (7.1) | 4 (26.7) | 12 (20.3) |
| Epistaxis | 1 (20.0) | 4 (26.7) | 2 (13.3) | 1 (7.1) | 2 (13.3) | 9 (15.3) |
| Diarrhoea | 0 | 1 (6.7) | 2 (13.3) | 0 | 2 (13.3) | 5 (8.5) |
| Platelet count increased | 0 | 0 | 0 | 0 | 4 (26.7) | 4 (6.8) |
| Vomiting | 0 | 1 (6.7) | 0 | 1 (7.1) | 2 (13.3) | 4 (6.8) |
| Ecchymosis | 0 | 0 | 0 | 3 (21.4) | 0 | 3 (5.1) |
| Nausea | 0 | 0 | 0 | 2 (14.3) | 1 (6.7) | 3 (5.1) |
| Pain in extremity | 0 | 0 | 2 (13.3) | 0 | 1 (6.7) | 3 (5.1) |
| Upper respiratory | 0 | 1 (6.7) | 0 | 2 (14.3) | 0 | 3 (5.1) |
| tract infection | | | | | | |
| Abdominal pain | 0 | 0 | 2 (13.3) | 0 | 0 | 2 (3.4) |
| Migraine | 0 | 0 | 0 | 0 | 2 (13.3) | 2 (3.4) |
| Petechiae | 1 (20.0) | 0 | 0 | 0 | 2 (13.3) | 2 (3.4) |

TABLE 9

| | | E5501 | E5501 | E5501 | E5501 | Total |
|---|---|---|---|---|---|---|
| MedDRA System | Placebo | 2.5 mg | 5 mg | 10 mg | 20 mg | E5501 |
| Organ Class | (N = 5) | (N = 15) | (N = 15) | (N = 14) | (N = 15) | (N = 59) |
| Preferred Term | n (%) | n (%) | n (%) | n (%) | n (%) | n (%) |
| Any Serious TEAE | 0 | 2 (13.3) | 0 | 1 (7.1) | 0 | 3 (5.1) |
| Blood and lymphatic | 0 | 1 (6.7) | 0 | 0 | 0 | 1 (1.7) |
| system disorders | | | | | | |
| Thrombocytopenia | 0 | 1 (6.7) | 0 | 0 | 0 | 1 (1.7) |
| Cardiac disorders | 0 | 0 | 0 | 1 (7.1) | 0 | 1 (1.7) |
| Myocardial infarction | 0 | 0 | 0 | 1 (7.1) | 0 | 1 (1.7) |
| Eye disorders | 0 | 0 | 0 | 1 (7.1) | 0 | 1 (1.7) |
| Retinal artery occlusion | 0 | 0 | 0 | 1 (7.1) | 0 | 1 (1.7) |
| Gastrointestinal disorders | 0 | 1 (6.7) | 0 | 0 | 0 | 1 (1.7) |
| Gastritis haemorrhagic | 0 | 1 (6.7) | 0 | 0 | 0 | 1 (1.7) |
| Infections and infestations | 0 | 0 | 0 | 1 (7.1) | 0 | 1 (1.7) |
| Pneumonia | 0 | 0 | 0 | 1 (7.1) | 0 | 1 (1.7) |
| Nervous system disorders | 0 | 0 | 0 | 1 (7.1) | 0 | 1 (1.7) |
| Transient ischaemic attack | 0 | 0 | 0 | 1 (7.1) | 0 | 1 (1.7) |

### Discussion

The results of this study demonstrate that E5501 at doses up to 20 mg increased platelet count with an early onset of action for a significant number of subjects who demonstrated a response. The study also provided information about the time-course of platelet response and safety over the 28-day treatment period in subjects with ITP.

The data indicated that the subjects who received E5501 ≧2.5 mg had a better response rate (13% to 80%) with increasing dose. Despite the sample size being relatively small, with only 14 or 15 subjects per E5501 dose and 5 subjects in the placebo group, the results demonstrated that E5501 20 mg had statistically significant superior efficacy compared to placebo (p=0.0036), as well as E5501 2.5 mg (p=0.0007)

E5501 increased platelet counts above the criterion response level relatively quickly. The majority (57.6%) of the subjects responded to a dose of ≧5 mg by day 7. Subjects treated with E5501 20 mg achieved a 93.3% response rate at day 7. Platelet counts were measured earlier, on day 3 in a total of 15 subjects and on day 5 in a total of 21 subjects. One of 13 subjects treated with E5501 responded by day 3, while 6 of 19 subjects treated with E5501 responded by day 5 (Table 10). In contrast, note of the 4 placebo-treated subjects responded at those times.

TABLE 10

| | | E5501 | E5501 | E5501 | E5501 | Total |
|---|---|---|---|---|---|---|
| Visit Category | Placebo | 2.5 mg | 5 mg | 10 mg | 20 mg | E5501 |
| Full Analysis Population | | | | | | |
| Total in the treatment | 5 | 15 | 15 | 14 | 15 | 59 |
| Day 3^{a} | | | | | | |
| Yes | 0 | 0 | 0 | 0 | 1 (25.0) | 1 (7.7) |
| No | 2 (100.0) | 4 (100.0) | 3 (100.0) | 2 (100.0) | 3 (75.0) | 12 (92.3) |
| Total | 2 | 4 | 3 | 2 | 4 | 13 |
| Day 5^{a} | | | | | | |
| Yes | 0 | 0 | 4 (66.7) | 1 (25.0) | 1 (20.0) | 6 (31.6) |
| No | 2 (100.0) | 4 (100.0) | 2 (33.3) | 3 (75.0) | 4 (80.0) | 13 (68.4) |
| Total | 2 | 4 | 6 | 4 | 5 | 19 |

^{a}Percentages are based on the total number of subjects with non-missing data in relevant treatment group.
OC = Observed Case.
Responder is defined as the proportion of subjects with Screening Visit B platelet count <30,000/mm3 who achieve a platelet count >=50,000/mm3, together with the proportion of subjects receiving steroids with a Screening Visit B platelet count >=30,000/mm3 but <50,000/mm3 who achieve a platelet count >=20,000/mm3 above their Screening Visit B platelet count.

The time course of platelet response generally showed an increase up to a maximum concentration on day 7 day followed by a decrease to a lower level at 28 days. The response rates were reduced by about 30% in the lower E5501 dose groups. The reduction in the response rate in the 20 mg dose group was only about 15%. Sustained platelet increases that met the criteria for positive response were observed for 0%, 0%, 28.6%, 41.7% and 76.9% of subjects on placebo, E5501 2.5 mg, 5 mg, 10 mg and 20 mg, respectively. This showed that the sustained platelet response to E5501 occurred in a dose-response fashion. It also suggests that a higher dose may be needed to maintain platelet response.

The overall response rate for the highest dose of E5501 tested in this study was 80-93%. In comparison, at day 7, 93% of subjects administered E5501 achieved a platelet response of 50,000/mm³, while only 25% of subjects receiving Nplate® achieved this increase in seven days (Kuter et al. Lancet (2008) 371:395-403), and 44-62% of subjects administered Promacta® achieved the same increase in eight days (see Bussel, et al. N. Engl. J. Med. (2007) 357:2237-47 and Bussel et al. Lancet (2009) 373:641-48). While Nplate® provided a 38% or a 56% durable plate response at 6 months to splenectomized or non-splenectomized subjects, respectively, in the current study, 66.7% of subjects who were refractory to splenectomy responded to 20 mg E5501. Although this study was not designed to determine the long term effects of E5501, a sustained platelet response for 28 days was observed in 76.9% of subjects.

The safety profile of E5501 was characterized by a similar proportion of subjects with any TEAEs across dose group. Although TEAEs were numerically highest in the 20 mg dose group, no serious TEAEs were reported in this group. Treatment emergent EAs that occurred in ≧5% of subjects receiving E5501 and at a rate at least twice that of placebo were diarrhea, contusion, platelet count excessively increased, vomiting, ecchymoses, nausea, pain in extremity and upper respiratory tract infection. Excessive platelet count increase occurred exclusively in the 20 mg dose group. Diarrhea, nausea and vomiting showed an inconsistent but potential dose-response; a total of 12 events occurred in 7 subjects. Most of these events occurred within the first 2 weeks of treatment, were mild in severity, were transient and completely resolved. The number of subjects with a TEAE leading to study drug withdrawal was small (n=4, 6.8%). The 20 mg dose group had the highest number of subjects with a TEAE leading to study drug withdrawal (n=2; 13.3%). Consistent with the drug's mechanism of action, both events were “platelet count excessively increased.” Three subjects had 6 serious TEAEs; there were no trends with respect to dose, system organ class or preferred term. Table 9 lists the serious TEAEs. Upon discontinuation of E5501, 3 subjects' platelet count decreased to below that of baseline, suggesting the potential for rebound thrombocytopenia.

### Conclusion

Based on these study results, E5501 demonstrated superior efficacy compared with placebo as measured by platelet response on day 28. This response was dose related. Platelet responses were observed in as early as 7 days following the start of treatment. Nearly 80% of subjects in the 20 mg group who completed 28 days of therapy maintained their platelet response for at least 3 weeks. E5501 was also well tolerated and had a favorable safety profile.

### Example 2

### A Phase 2, Parallel Group, Rollover Study of E5501 in Subjects with Chronic Idiopathic Thrombocytopenia Purpura (ITP) who Completed 28 Days of Study Treatment

### Introduction

This study was a multicenter, parallel-group, rollover study of E5501 in subjects with chronic ITP who were enrolled in and completed 28 days' of study treatment. All subjects who completed the previous study and otherwise met the eligibility criteria for this rollover protocol were enrolled.

### Summary of Study Design

Subjects who rolled over from the previous study were treated for an additional 6 months after completing previous study. Subjects were initially separated into 2 groups, namely, subjects who had responded (e.g., responders) or did not respond (e.g., non-responders) to E5501 in the previous study.

Initial dose: Subjects who met the efficacy response in the previous study continued to receive their previous blinded dose at entry into this study. However, 2 subject who did not meet the efficacy response in the previous study (e.g., non-responders), initially received E5501 10 mg once daily in an open-label fashion regardless of study drug or dose received in the previous study.

Dose modification: The study protocol permitted dose escalation of E5501 for subjects who did not achieve a platelet response during treatment. Dose titration was performed in an open label fashion in 10 mg increments as follows:
Nonresponders, who received initial doses of open-label E5501 10 mg in the previous study, could have their dose increased in 10 mg increments every 14 days to a maximum of 40 mg/day in the absence of platelet response. If the subject did not have a platelet response at 40 mg/day, then study drug was to be permanently discontinued.
Responders could receive up to 2 open-label dose increases in 10 mg increments (e.g., total additional open-label dose of 20 mg/day) every 14 days in addition to their double-blind dose. If the subject did not respond to this higher dose, then study drug was to be permanently discontinued.

Subjects were also permitted to reduce their dose of ITP-directed concomitant therapy (e.g., steroids). The overall goal of any dose modification was to maintain the subject's peripheral platelet count above 50,000/mm³ and to decrease the need for ITP-directed concomitant medications (if applicable). During any dose modification of study drug or ITP-directed concomitant medications, platelet counts were collected every week. After platelet counts stabilized, platelet count collection reverted to a biweekly schedule.

Assessments: Study subjects were evaluated for safety and efficacy (including platelet count) on day 1 and then biweekly while receiving treatment. Subjects receiving open-label E5501 also had a Day 7 evaluation.

### Study Objectives

The primary objective of this study was to assess the safety and tolerability of E5501 administered for an additional 6 months in subjects with chronic ITP who completed 28 days of previous treatment with E5501. The secondary objectives were to evaluate markers of effectiveness of E5501.

### Study Populations

Two primary analysis populations were used to summarize safety and effectiveness:
Safety population: all subjects who received at least 1 dose of study drug and had at least 1 post-treatment safety assessment in either the previous or this study. All safety analyses were performed using this population.
Full Analysis Set (FAS): all subjects who participated in both studies and who had at least one post-treatment effectiveness assessment in this study. All efficacy analyses were performed using this population.

An additional analysis population of medical interest was defined as follows:
Sufficient Exposure (SE) population: all subjects in the FAS excluding those who did not attain 12 weeks of treatment and those who did not have an upward dose titration when they required one based on inadequate platelet response, defined as a platelet count of <50,000/mm³.

It is important to note that the protocol permitted upward dose titration in the event of platelet nonresponse, at which time 18 subjects had already been enrolled in this study. Seven of the 18 subjects were nonresponders in the previous study and 3 had been withdrawn from this study before Protocol Amendment 4 was implemented.

### Study Endpoints

Primary Endpoint: The primary endpoint was to assess the safety and tolerability to E5501, including AEs and clinical laboratory parameters, over the 6 additional months of treatment.

Secondary Endpoints: The secondary endpoints were markers of effectiveness, including:
1. Changes in platelet count from baselines of the previous study and of this study by study visit; and
2. Durable platelet response, defined as subjects who had at least 3 platelet count assessments in the last 14 weeks of the 24-week treatment period, and whose platelet count was at a response level for at least 75% of the time in the last 14 weeks during the 24-week treatment period, with no rescue medication used during the 24-week treatment period. Platelet response level was defined as ≧50,000/mm³ for subjects whose baseline platelet count was <30,000/mm³ in the previous study or an increase in baseline platelet count of ≧20,000/mm³ for subjects receiving steroids whose baseline platelet count in the previous study was ≧30,000/mm³ but <50,000/mm³;
3. Transient platelet response, defined as achievement of a response level for any 4 consecutive weeks during the 24-week treatment period without durable platelet response in the absence of any rescue medication;
4. Overall platelet response (durable platelet response plus transient platelet response);
5. Maintenance of platelet response, defined as subjects who were responders in the previous study (e.g., responders on day 28 of the previous study) who achieved a durable response in this study and had no upward dose titration of E5501 during this study;
6. Permanent discontinuation of steroid use, defined as subjects who used steroid medication within the 2-week period before their first dose of study drug in this study, and for whom concomitant steroid medication was discontinued at least 8 weeks before the end of the treatment period;
7. Reduction of steroid use by at least 50%.

### Analysis Methods

For a more comprehensive understanding of the safety profile of E5501, the safety analysis was performed for the 64 subjects who received at least 1 dose of E5501 using the combined safety data from both studies. In this way, safety data reflect each subject's complete duration of exposure to E5501 starting with lead-in the previous study and continuing into the current study.

Efficacy results focused on long-term treatment effectiveness and were based on the 53 subjects who received E5501 during the 6-months of the current study (excluding data from the previous study). In addition, a sensitivity analysis, which also included subjects who received E5501 10 mg in the previous study and who did not participate in this study, was performed on the effectiveness results to facilitate dose selection for future studies. Eleven subjects from the previous study did not rollover into this study (Table 12). Data for 2 of the 11 subjects were considered to be relevant in determining durable platelet response because these subjects received E5501 10 mg; (e.g., the same dose of E5501 that was planned as the starting dose in Phase 3 studies. Both subjects were classified as nonresponders for durable response (e.g., they did not enroll in this study and, therefore, did not have effectiveness data during the last 14 weeks of the 6-month treatment period in this study). Data for the remaining 9 subjects were not included in the sensitivity analysis; 7 subjects received a dose of E5501 lower than 10 mg (2.5 mg [n=3] or 5 mg [n=4]) in the previous study and 2 subjects had an excessive increase in platelet count (e.g., >500,000/mm³) during treatment with E5501 20 mg in the previous study. Thus, the sensitivity analysis was based on a total of 55 subjects (e.g., 53 subjects who participated in the current study plus the 2 nonresponders who received E5501 10 mg in the previous study but who did not enroll in Study 501-CL-004).

Following Protocol Amendment 4, the current study contained a flexible dose regimen; therefore, this study was considered to have an open-label, uncontrolled, single-arm design. Because of these limitations in study design, two exploratory grouping methods were proposed for the efficacy and safety analyses:
Grouping method A was based on average daily dose received during the combined active treatment periods of both studies. Mean daily dose groups were defined as follows: lower ⅓ (subjects who received <10 mg/day), middle ⅓ (subjects who received ≧10-<16 mg) and upper ⅓ (subjects who received ≧16 mg). This was the method used to assess safety.
Grouping method B was based on both treatment received and day 28 platelet response to study drug in Study the previous study. This was the method used for the analysis of efficacy for the FAS and the SE population.

### Results

Subject Disposition: Sixty-four subjects enrolled in the first study, and among them, 53 subjects enrolled in the rollover study, (e.g., the current study). The number and percentage of subjects who completed the study or were withdrawn prematurely are summarized in Table 11 and Table 12 for the safety population and FAS, respectively. In addition, the number and percentage of subjects who permanently withdrew are summarized by reason for discontinuation, grouped by response status and by average daily dose level groups.

TABLE 11

| | Lower ⅓ | | Middle ⅓ | | Upper ⅓ | | | |
|---|---|---|---|---|---|---|---|---|
| | Mean Daily | | Mean Daily | | Mean Daily | | | |
| | Dose Level | | Dose Level | | Dose Level | | E5501 | |
| Population Status | (2.5-<8.85 mg) | | (≧8.85-<13.5 mg) | | (≧13.5-≦25.7 mg) | | Total | |
| Safety Population, N | 22 | | 22 | | 20 | | 64 | |
| Completed 501-CL-003 and | 11 | (50.0) | 13 | (59.1) | 11 | (55.0) | 35 | (54.7) |
| 501-CL-004, n (%) | | | | | | | | |
| Completed 501-CL-003 but did not | 4 | (18.2) | 0 | | 0 | | 4 | (6.3) |
| rollover into 501-CL-004, n (%) | | | | | | | | |
| Discontinued 501-CL-003 and did not | 3 | (13.6) | 2 | (9.1) | 2 | (10.0) | 7 | (10.9) |
| rollover into 501-CL-004, n (%) | | | | | | | | |
| Discontinued 501-CL-004, n (%) | 4 | (18.2) | 7 | (31.8) | 7 | (35.0) | 18 | (28.1) |
| Primary Reason for Discontinuation | | | | | | | | |
| in 501-CL-003: | | | | | | | | |
| PC increase to ≧500,000/mm³ | 0 | | 0 | | 2 | (10.0) | 2 | (3.1) |
| Withdrew Consent | 1 | (4.5) | 1 | (4.5) | 0 | | 2 | (3.1) |
| Adverse Event or SAE | 1 | (4.5) | 1 | (4.5) | 0 | | 2 | (3.1) |
| Other | 1 | (4.5) | 0 | | 0 | | 1 | (1.6) |
| Primary Reason for Discontinuation | | | | | | | | |
| in 501-CL-004: | | | | | | | | |
| Adverse Event or SAE | 2 | (9.1) | 2 | (9.1) | 1 | (5.0) | 5 | (7.8) |
| Noncompliance with Protocol | 0 | | 0 | | 1 | (5.0) | 1 | (1.6) |
| PC increase to ≧500,000/mm³ | 1 | (4.5) | 0 | | 0 | (0.0) | 1 | (1.6) |
| Laboratory Abnormality | 0 | | 0 | | 0 | | 0 | |
| Withdrew Consent | 0 | | 1 | (4.5) | 2 | (10.0) | 3 | (4.7) |
| Investigator Decision | 1 | (4.5) | 3 | (13.6) | 2 | (10.0) | 6 | (9.4) |

| | | | | | | | |
|---|---|---|---|---|---|---|---|
| Other | 0 | 1 | (4.5) | 1 | (5.0) | 2 | (3.1) |

PC = platelet count
Grouping method A was based on the mean daily dose received during the combined active treatment period, defined as the period between the day of first dose and the day of last dose of active study drug (excluding tapering period.
The Safety population included all subjects who received at least 1 does of study drug and had at least 1 post-treatment safety assessment in Studies either the previous or current study.

TABLE 12

| | | | | | E5501 | |
|---|---|---|---|---|---|---|
| Population | Responders^{a} | | Nonresponders^{a} | | Total | |
| Full Analysis | 25 | | 28 | | 53 | |
| Set, N | | | | | | |
| Completed | 20 | (80.0) | 15 | (53.6) | 35 | (66.0) |
| 501-CL-004, n (%) | | | | | | |
| Discontinued | 5 | (20.0) | 13 | (46.4) | 18 | (34.0) |
| 501-CL-004, n (%) | | | | | | |
| Primary Reason for | | | | | | |
| Discontinuation | | | | | | |
| Adverse Event | 2 | (8.0) | 3 | (10.7) | 5 | (9.4) |
| or SAE | | | | | | |
| Noncompliance | 0 | | 1 | (3.6) | 1 | (1.9) |
| with Protocol | | | | | | |
| PC increase | 0 | | 1 | (3.6) | 1 | (1.9) |
| to ≧500,000/mm³ | | | | | | |
| Withdrew Consent | 1 | (4.0) | 2 | (7.1) | 3 | (5.7) |
| Investigator Decision | 1 | (4.0) | 5 | (17.9) | 6 | (11.3) |
| Other | 1 | (4.0) | 1 | (3.6) | 2 | (3.8) |

PC = platelet count
Grouping method B was based on the dose of study drug received in the previous study and platelet response to E5501 on day 28 of the previous study.
Full Analysis set included all subjects who enrolled in the current study and who provided adequate data to derive at least 1 efficacy assessment in the current study.
^{a}Subjects' response status based on Day 28 of the previous study.

Fifty-three subjects (approximately 83%) in the previous study rolled over into the current study. Of the 11 subjects who did not continue into this study, 7 were ineligible for enrollment after having withdrawn prematurely from the previous study. The remaining 4 subjects completed the previous study but chose not to participate in the current study. Among the 53 subjects who continued into the current study, approximately two-thirds (66%) of subjects completed the 6-month treatment period, with the majority (80%) of responders and slightly more than half (53.6%) of non-responders in the FAS completing the study. For the lower ⅓, middle ⅓, and upper ⅓ mean daily dose level groups, 50%, 59.1% and 55% of subjects, respectively, completed both studies. Eighteen subjects withdrew prematurely from the current study: 4, 7, and 7 subjects in the lower, middle, and upper ⅓ mean daily dose level groups, respectively (Table 1). Five subjects had been responders and 13 non-responders in the previous study (Table 2). Of the 18 subjects who withdrew, 5 subjects (2 responders and 3 non-responders, or n=2, 2 and 1 for the lower, middle, and upper ⅓ dose level groups) withdrew from the current study due to AEs, while only 1 subject (non-responder, lower ⅓ dose level) withdrew due to an excessively increased platelet count (≧500,000/mm³).

### Subject Demographics and Baseline Characteristics

Subject demographics and baseline characteristics are summarized in Table 13 and Table 14 for the Safety population and FAS, respectively. Continuous variables such as age, weight and height at screening, baseline platelet count category (≦15,000/mm³ vs. ≧15,000/mm³), history of splenectomy, number of lines of prior therapy, and prior steroid use are summarized by descriptive statistics (m, mean, standard deviation, median, minimum and maximum). Categorical variables such as sex, race and reproductive status are summarized by number and percentage. In general, demographic and baseline characteristics were comparable among subjects grouped by responder status in the previous study and by E5501 average daily dose level in the previous study and the current study as shown in Table 13 and Table 14, respectively.

TABLE 13

| | Lower ⅓ | | Middle ⅓ | | Upper ⅓ | | | |
|---|---|---|---|---|---|---|---|---|
| | Mean Daily | | Mean Daily | | Mean Daily | | | |
| | Dose Level | | Dose Level | | Dose Level | | E5501 | |
| Category | (2.5-<8.85 mg) | | (≧8.85-<13.5 mg) | | (≧13.5-≦25.7 mg) | | Total | |
| Age (Year) | | | | | | | | |
| n | 22 | | 22 | | 20 | | 64 | |
| Mean (SD) | 51.1 | (20.9) | 56.0 | (15.4) | 49.6 | (17.3) | 52.3 | (18.0) |
| Median | 54.5 | | 58.0 | | 52.5 | | 55.5 | |
| Min, Max | 18, 81 | | 23, 82 | | 22, 77 | | 18, 82 | |
| Sex, n (%) | | | | | | | | |
| Male | 10 | (45.5) | 8 | (36.4) | 6 | (30.0) | 24 | (37.5) |
| Female | 12 | (54.5) | 14 | (63.6) | 14 | (70.0) | 40 | (62.5) |
| Race, n (%) | | | | | | | | |
| Black, African Heritage | 1 | (4.5) | 2 | (9.1) | 2 | (10.0) | 5 | (7.8) |
| Asian | 3 | (13.6) | 0 | | 1 | (5.0) | 4 | (6.3) |
| Hispanic | 3 | (13.6) | 1 | (4.5) | 1 | (5.0) | 5 | (7.8) |
| White | 14 | (63.6) | 19 | (86.4) | 15 | (75.0) | 48 | (75.0) |
| Other | 1 | (4.5) | 0 | | 1 | (5.0) | 2 | (3.1) |
| Height (cm) | | | | | | | | |
| n | 20 | | 20 | | 17 | | 57 | |
| Mean (SD) | 167.6 | (11.4) | 168.8 | (11.5) | 168.7 | (11.6) | 168.3 | (11.3) |
| Median | 168.0 | | 170.0 | | 165.0 | | 168.0 | |
| Min, Max | 152, 191 | | 150, 193 | | 155, 196 | | 150, 196 | |
| Weight (kg) | | | | | | | | |
| n | 22 | | 21 | | 19 | | 62 | |
| Mean (SD) | 77.8 | (17.6) | 81.5 | (16.8) | 80.8 | (16.8) | 80.0 | (16.9) |
| Median | 76.0 | | 78.0 | | 74.0 | | 76.5 | |
| Min, Max | 53, 109 | | 57, 124 | | 59, 122 | | 53, 124 | |
| History of Splenectomy | | | | | | | | |
| Yes | 8 | (36.4) | 7 | (31.8) | 5 | (25.0) | 20 | (31.3) |
| No | 14 | (63.6) | 15 | (68.2) | 15 | (75.0) | 44 | (68.8) |
| Baseline Steroid Usage | | | | | | | | |
| Yes | 10 | (45.5) | 10 | (45.5) | 11 | (55.0) | 31 | (48.4) |
| No | 12 | (54.5) | 12 | (54.5) | 9 | (45.0) | 33 | (51.6) |
| Baseline Platelet | | | | | | | | |
| Count Category | | | | | | | | |
| ≦15 K/mm³ | 4 | (18.2) | 8 | (36.4) | 6 | (30.0) | 18 | (28.1) |
| >15 K/mm³ | 18 | (81.8) | 14 | (63.6) | 14 | (70.0) | 46 | (71.9) |
| Number of Lines of | | | | | | | | |
| Prior Therapies | | | | | | | | |
| <3 | 11 | (50.0) | 6 | (27.3) | 7 | (35.0) | 24 | (37.5) |
| ≧3 | 11 | (50.0) | 16 | (72.7) | 13 | (65.0) | 40 | (62.5) |

Grouping method A was based on the mean daily dose received during the combined active treatment period, defined as the period between the day of first dose and the day of last dose of active study drug (excluding tapering period.
The Safety population included all subjects who received at least 1 dose of study drug and had at least 1 post-treatment safety assessment in either study.

TABLE 14

| | | | | | E5501 Total | |
|---|---|---|---|---|---|---|
| Category | Responders^{a} | | Nonresponders^{a} | | (N = 53) | |
| Age (Year) | | | | | | |
| n | 25 | | 28 | | 53 | |
| Mean (SD) | 48.4 | (17.6) | 51.6 | (19.0) | 50.1 | (18.3) |
| Median | 48.0 | | 53.0 | | 51.0 | |
| Min, Max | 18, 77 | | 20, 81 | | 18, 81 | |
| Sex, n (%) | | | | | | |
| Male | 8 | (32.0) | 7 | (25.0) | 15 | (28.3) |
| Female | 17 | (68.0) | 21 | (75.0) | 38 | (71.7) |
| Race, n (%) | | | | | | |
| Black, African | 3 | (12.0) | 2 | (7.1) | 5 | (9.4) |
| Heritage | | | | | | |
| Asian | 1 | (4.0) | 0 | | 1 | (1.9) |
| Hispanic | 2 | (8.0) | 3 | (10.7) | 5 | (9.4) |
| White | 17 | (68.0) | 23 | (82.1) | 40 | (75.5) |
| Other | 2 | (8.0) | 0 | | 2 | (3.8) |
| Height (cm) | | | | | | |
| n | 21 | | 25 | | 46 | |
| Mean (SD) | 166.8 | (9.86) | 166.3 | (10.45) | 166.5 | (10.1) |
| Median | 165.0 | | 165.0 | | 165.0 | |
| Min, Max | 150, 185 | | 152, 193 | | 150, 193 | |
| Weight (kg) | | | | | | |
| n | 24 | | 27 | | 51 | |
| Mean (SD) | 83.3 | (17.32) | 73.1 | (13.59) | 77.9 | (16.13) |
| Median | 78.5 | | 69.0 | | 74.0 | |
| Min, Max | 57, 124 | | 53, 104 | | 53, 124 | |
| History of | | | | | | |
| Splenectomy | | | | | | |
| Yes | 7 | (28.0) | 10 | (35.7) | 17 | (32.1) |
| No | 18 | (72.0) | 18 | (64.3) | 36 | (67.9) |
| Baseline | | | | | | |
| Steroid Usage | | | | | | |
| Yes | 14 | (56.0) | 11 | (39.3) | 25 | (47.2) |
| No | 11 | (44.0) | 17 | (60.7) | 28 | (52.8) |
| Baseline Platelet | | | | | | |
| Count Category | | | | | | |
| ≦15 K/mm³ | 6 | (24.0) | 9 | (32.1) | 15 | (28.3) |
| >15 K/mm³ | 19 | (76.0) | 19 | (67.9) | 38 | (71.7) |
| Number of Lines of | | | | | | |
| Prior Therapies | | | | | | |
| <3 | 12 | (48.0) | 6 | (21.4) | 18 | (34.0) |
| ≧3 | 13 | (52.0) | 22 | (78.6) | 35 | (66.0) |

Grouping method B was based on dose of E5501 received in the previous study and platelet response to study drug on Day 28 of the previous study.
Full Analysis set included all subjects who provided adequate data to derive at least 1 efficacy assessment in the current study.
^{a}Subjects' response status based on day 28 of the previous study.

### Effectiveness Results

In this summary, the effectiveness endpoints of clinical interest included durable, transient, and overall response rates, and changes in concomitant ITP drug usage.

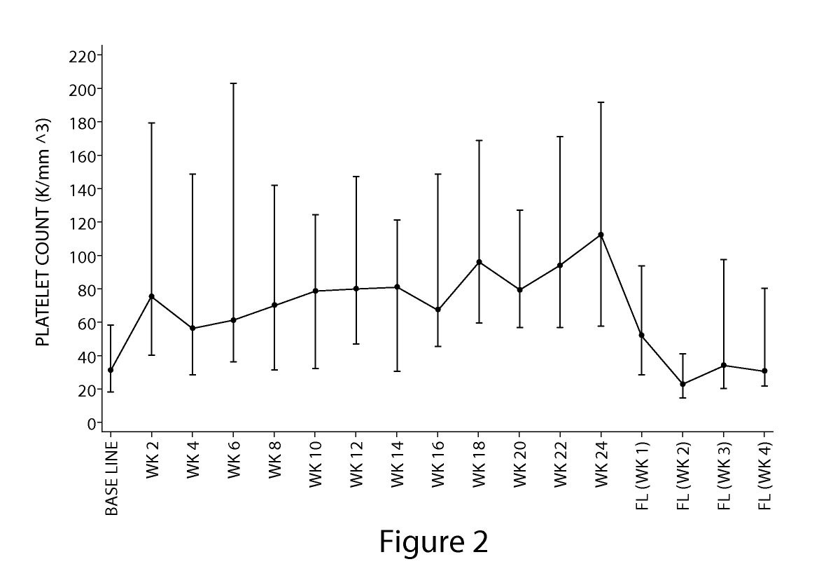

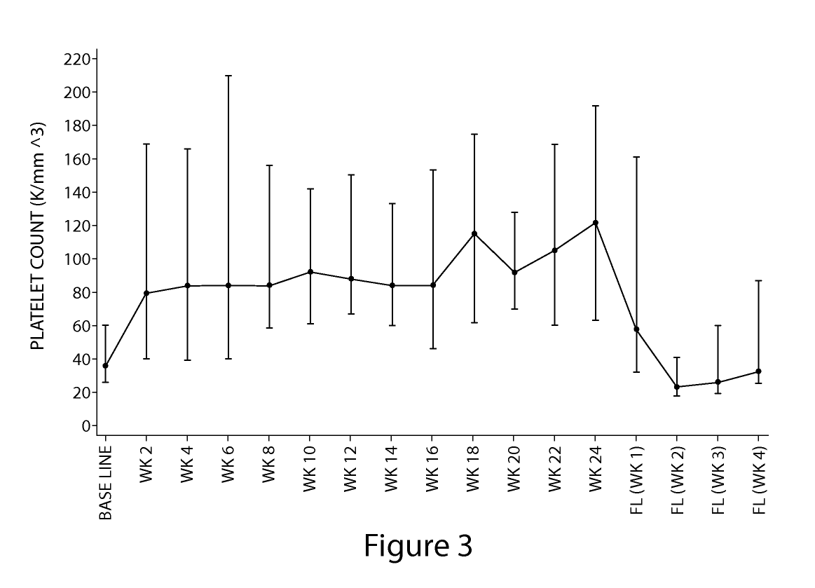

For the FAS, the effectiveness of 6 months' treatment with E5501 was demonstrated among both subjects who were responders and those who were non-responders in the previous study (Table 15). Following treatment with E5501, platelet counts were markedly increased from baseline as shown in FIG. 2 for the FAS and FIG. 3 for the SE population. The observed durable response rate during the 6-month treatment period was 52.8% for all subjects, 72.0% for responders, and 35.7% for non-responders. A sensitivity analysis based on 55 subjects yielded a durable platelet response estimate of 50.9% for all subjects (n=28/55), 72% for responders (n=18/25), and 33.3% for non-responders (n=10/30).

Similarly, the overall platelet response rate was 75.5% for all subjects, 88.0% for responders, and 64.3% for nonresponders (Table 15). However, the overall response rates for non-responders who had been receiving placebo, E5501 2.5 mg, or E5501 5 mg in the previous study and who received open-label E5501 10 mg in the current study were 80%, 70%, and 80%, respectively (data not shown). These were comparable to the overall response rate of 71.4% for responders who were receiving double-blind E5501 10 mg. Five subjects did not respond to E5501 10 mg in the previous study and all 5 non-responders were treated with a starting dose of open-label E5501 10 mg in the current study. Two of these subjects responded when treated with E5501 20 mg in the current study. Another 2 subjects never received an upward dose titration even though they required it per protocol, while the fifth subject had platelet counts as high as >1,000,000/mm³ and as low as <10,000/mm³ after receiving multiple dose adjustments. The number of subjects in each of these groups was small; therefore, a definitive conclusion cannot be made.

For the SE population, the durable and overall platelet response rates increased, particularly among non-responders, 52.4% of whom required upward dose titration, but did not receive it (Table 15). Durable platelet response rates for the SE population were 75.0% for all subjects, 85.7% for responders, and 60.0% for non-responders.

TABLE 15

| | | | Non- | | E5501 | |
|---|---|---|---|---|---|---|
| Category | Responders^{a} | | Responders^{a} | | Total | |
| Full Analysis Set | 25 | | 28 | | 53 | |
| Durable Platelet | | | | | | |
| Response | | | | | | |
| Yes, n (%) | 18 | (72.0) | 10 | (35.7) | 28 | (52.8) |
| No, n (%) | 7 | (28.0) | 18 | (64.3) | 25 | (47.2) |
| Transient Platelet | | | | | | |
| Response | | | | | | |
| Yes, n (%) | 4 | (16.0) | 8 | (28.6) | 12 | (22.6) |
| No, n (%)^{b} | 21 | (84.0) | 20 | (71.4) | 41 | (77.4) |
| With Overall | | | | | | |
| Response | | | | | | |
| Yes, n (%) | 22 | (88.0) | 18 | (64.3) | 40 | (75.5) |
| No, n (%) | 3 | (12.0) | 10 | (35.7) | 13 | (24.5) |
| Sufficient Exposure | 21 | | 15 | | 36 | |
| Population, N | | | | | | |
| Durable Platelet | | | | | | |
| Response | | | | | | |
| Yes, n (%) | 18 | (85.7) | 9 | (60.0) | 27 | (75.0) |
| No, n (%) | 3 | (14.3) | 6 | (40.0) | 9 | (25.0) |
| Transient Platelet | | | | | | |
| Response | | | | | | |
| Yes, n (%) | 3 | (14.3) | 4 | (26.7) | 7 | (19.4) |
| No, n (%) | 18 | (85.7) | 11 | (73.3) | 29 | (80.6) |
| With Overall | | | | | | |
| Response | | | | | | |
| Yes, n (%) | 21 | (100.0) | 13 | (86.7) | 34 | (94.4) |

| | | | | | |
|---|---|---|---|---|---|
| No, n (%) | 0 | 2 | (13.3) | 2 | (5.6) |

FAS = full analysis set; SE = sufficient exposure
Grouping method B was based on dose of E5501 received in the previous study and platelet response to study drug on day 28 in the previous study.
Full Analysis set included all subjects who provided adequate data to derive at least 1 efficacy assessment in the current study.
^{a}Subjects' response status based on day 28 of the current study.
^{b}Subjects in ‘no’ category for transient platelet response includes subjects who achieved a durable response.

Approximately one-third (33.3%) of subjects who used concomitant steroids were able to permanently discontinue using steroids regardless of responder status as shown in Table 16. Similarly, more than half (54.2%) of subjects who used concomitant steroids were able to decrease their concomitant steroid use by at least 50% regardless of responder status.

In the subgroup analyses, the data indicated that higher overall and durable platelet response rates were observed in subjects with a baseline platelet count >15,000/mm³, those who received <3 lines of prior therapy for ITP, and those who had not had a splenectomy (Table 17).

TABLE 16

| | | | E5501 |
|---|---|---|---|
| Category | Responders^{a} | Nonresponders^{a} | Total |
| Permanently discontinued | | | |
| steroid use | | | |
| Yes, n (%) | 4 (33.3) | 4 (33.3) | 8 (33.3) |
| No, n (%) | 8 (66.7) | 8 (66.7) | 16 (66.7) |
| Total | 12 | 12 | 24 |
| Decreased use of | | | |
| concomitant steroid | | | |
| medication by ≧50% | | | |
| Yes, n (%) | 7 (58.3) | 6 (50.0) | 13 (54.2) |
| No, n (%) | 5 (41.7) | 6 (50.0) | 11 (45.8) |
| Total | 12 | 12 | 24 |

Only subjects who had been taking steroids at baseline were included in this analysis. The denominator of the proportion is the number of subjects who used steroids at baseline (e.g., the 2-week period before the date of first dose in the current study).
Grouping method B was based on dose of E5501 received in the previous study and platelet response to E5501 on day 28 in the previous study.
Full Analysis set included all subjects who provided adequate data to derive at least 1 efficacy assessment in the current study.
^{a}Subjects' response status was based on platelet count results on day 28 of the current study.

TABLE 17

| | Durable Platelet Response | | | Overall Platelet Response | | |
|---|---|---|---|---|---|---|
| | | Non- | E5501 | | Non- | E5501 |
| Category | Responders^{a} | Responders^{a} | Total | Responders^{a} | Responders^{a} | Total |
| No History of Splenectomy, | | | | | | |
| N assessed | 18 | 18 | 36 | 18 | 18 | 36 |
| n (%) with response | 14 (77.8) | 9 (50.0) | 23 (63.9) | 17 (94.4) | 13 (72.2) | 30 (83.3) |
| History of Splenectomy, | | | | | | |
| N assessed | 7 | 10 | 17 | 7 | 10 | 17 |
| n (%) with response | 4 (57.1) | 1 (10.0) | 5 (29.4) | 5 (71.4) | 5 (50.0) | 10 (58.8) |
| Baseline PC ≦15K/mm³, | | | | | | |
| N assessed | 6 | 9 | 15 | 6 | 9 | 15 |
| n (%) with response | 3 (50.0) | 1 (11.1) | 4 (26.7) | 4 (66.7) | 3 (33.3) | 7 (46.7) |
| Baseline PC >15K/mm³, | | | | | | |
| N assessed | 19 | 19 | 38 | 19 | 19 | 38 |
| n (%) with response | 15 (78.9) | 9 (47.4) | 24 (63.2) | 18 (94.7) | 15 (78.9) | 33 (86.8) |
| <3 Lines of Prior Therapy, | | | | | | |
| N assessed | 12 | 6 | 18 | 12 | 6 | 18 |
| n (%) with response | 9 (75.0) | 3 (50.0) | 12 (66.7) | 11 (91.7) | 4 (66.7) | 15 (83.3) |
| ≧3 Lines of Prior Therapy, | | | | | | |
| N assessed | 13 | 22 | 35 | 13 | 22 | 35 |
| n (%) with response | 9 (69.2) | 7 (31.8) | 16 (45.7) | 11 (84.6) | 14 (63.6) | 25 (71.4) |

PC = platelet count
Grouping method B was based on dose of E5501 received in the previous study and platelet response to E5501 on day 28 in the previous study.
Full Analysis set included all subjects who provided adequate data to derive at least 1 efficacy assessment in the current study.
^{a}Response status based on day 28 of the previous study.

In an additional data summary, the status of E5501 upward dose titration was examined to explore the appropriate dose needed to achieve a platelet response for the FAS. Upward titration was required when 2 consecutive platelet counts were <50,000/mm³. The results indicated that most of the responders in the previous study (who were maintained on the dose to which they had been assigned) did not require upward dose titration in this study, except those who had been assigned to receive E5501 2.5 mg once daily. Meanwhile, almost half of the non-responders in the previous study (who initiated treatment in the present study with open label E5501 10 mg once daily per protocol) did require an upward dose titration during the 6-month treatment period (Table 18). That is, 5 (20%) of the 25 responders required upward dose titration, while 21 (75%) of 28 non-responders required upward dose titration during the 6-month treatment period of this study. Of the 26 subjects who required upward dose titration, 3 responders (60%) and 10 non-responders (47.6%) actually received it. For those subjects who required and did receive a dose titration, 10 (76.9%) of 13 subjects achieved an overall platelet response after the titration. These results suggest that, at least for some non-responders in the previous study, an overall platelet response could be achieved when the subject is given higher doses of E5501.

Overall, the findings based on a summary of the efficacy data in this study support the conclusion that E5501 is an effective treatment for ITP, and these efficacy results are consistent with the findings observed in the previous study.

TABLE 18

| | 501-CL-003 | | 501-CL-003 | | | |
|---|---|---|---|---|---|---|
| | Responders^{a} | | Nonresponders^{a} | | Total | |
| | (N = 25) | | (N = 28) | | (N = 53) | |
| Category | n (%) of subjects with: | | | | | |
| Upward titration | 5 | (20.0) | 21 | (75.0) | 26 | (49.0) |
| required | | | | | | |
| Upward titration | 3/5 | (60.0) | 10/21 | (47.6) | 13/26 | (50.0) |
| required and | | | | | | |
| implemented^{b} | | | | | | |
| Upward titration | 3/3 | (100.0) | 7/10 | (70.0) | 10/13 | (76.9) |
| required and | | | | | | |
| implemented; subject | | | | | | |
| achieved overall | | | | | | |
| response^{b} | | | | | | |

Grouping method B was based on dose of E5501 received in the previous study and platelet response to E5501 on day 28 in the previous study.
Full Analysis set included all subjects who provided adequate data to derive at least 1 efficacy assessment in the current study.
^{a}Response status based on Day 28 of Study 501-CL-003.
^{b}Denominator based on number of subjects who required upward titration and for whom the titration was implemented.

### Safety Results

Overall Adverse Events. In this summary of safety results, the focus is on TEAEs—overall and by preferred term (PT) during the combined first and second treatment periods as well as serious TEAEs (PT). The primary focus is on summary statistics for the entire safety population and provides an overall safety profile for E5501. The summary statistics for each group, e.g., lower ⅓, middle ⅓ and upper ⅓ average daily dose levels, is secondary and provides further safety information across the 3 dose levels.

Sixty-two of the 64 subjects (96.9%) experienced one or more TEAEs. For 41 of the 62 subjects (64.1%), the event was either possibly or probably treatment related (Table 19). Seven subjects had TEAEs that led to premature discontinuation of study treatment and 8 subjects had TEAEs that led to interruption of study treatment. For 1 of these 8 subjects, treatment was discontinued due to an increased platelet count >500,000/mm³. No deaths occurred during the study.

TABLE 19

| | Lower ⅓ | | Middle ⅓ | | Upper ⅓ | | | |
|---|---|---|---|---|---|---|---|---|
| | Mean Daily | | Mean Daily | | Mean Daily | | | |
| | Dose Level | | Dose Level | | Dose Level | | Total | |
| AE Category | (2.5-<8.85 mg) | | (≧8.85-<13.5 mg) | | (≧13.5-≦25.7 mg) | | E5501 | |
| Number (%) of subjects with: | (N = 22) | | (N = 22) | | (N = 20) | | (N = 64) | |
| Any TEAE | 20 | (90.9) | 22 | (100.0) | 20 | (100.0) | 62 | (96.9) |
| Severe (grade 3-4) TEAEs | 4 | (18.2) | 5 | (22.7) | 7 | (35.0) | 16 | (25.0) |
| Suspected drug-related AEs | 13 | (59.1) | 15 | (68.2) | 13 | (65.0) | 41 | (64.1) |
| Serious TEAE | 1 | (4.5) | 3 | (13.6) | 1 | (5.0) | 5 | (7.8) |
| Serious treatment-related TEAEs | 1 | (4.5) | 1 | (4.5) | 0 | | 2 | (3.1) |
| Withdrawal due to TEAE | 3 | (13.6) | 2 | (9.1) | 2 | (10.0) | 7 | (10.9) |
| Dose interruption due to TEAE | 3 | (13.6) | 3 | (13.6) | 2 | (10.0) | 8 | (12.5) |

| | | | | |
|---|---|---|---|---|
| Deaths | 0 | 0 | 0 | 0 |

TEAE = treatment-emergent adverse event
Grouping method A was based on the mean daily dose received during the combined active treatment period, defined as the period between the day of first dose and the day of last dose of E5501 (excluding tapering period.
The Safety population included all subjects who received at least 1 dose of E5501 and had at least 1 posttreatment safety assessment in either the previous or current study.

Most TEAEs were grade 1 or 2, transient, and completely resolved. Fatigue (34.4%), headache (32.8%), epistaxis (23.4%), contusion (18.8%), arthralgia (14.1%), diarrhea (14.1%), gingival bleeding (10.9%), and vomiting (10.9%) were the only AEs occurring in ≧10% of E5501-treated subjects overall (Table 20).

TABLE 20

| | Lower ⅓ | | Middle ⅓ | | Upper ⅓ | | | |
|---|---|---|---|---|---|---|---|---|
| | Mean Daily | | Mean Daily | | Mean Daily | | | |
| | Dose Level | | Dose Level | | Dose Level | | Total | |
| | (2.5-<8.85 mg) | | (≧8.85-<13.5 mg) | | (≧13.5-≦25.7 mg) | | E5501 | |
| | (N = 22) | | (N = 22) | | (N = 20) | | (N = 64) | |
| MedDRA Preferred Term | n (%) of subjects with: | | | | | | | |
| Any TEAE | 20 | (90.9) | 22 | (100.0) | 20 | (100.0) | 62 | (96.9) |
| Fatigue | 8 | (36.4) | 7 | (31.8) | 7 | (35.0) | 22 | (34.4) |
| Headache | 4 | (18.2) | 9 | (40.9) | 8 | (40.0) | 21 | (32.8) |
| Epistaxis | 4 | (18.2) | 3 | (13.6) | 8 | (40.0) | 15 | (23.4) |
| Contusion | 3 | (13.6) | 3 | (13.6) | 6 | (30.0) | 12 | (18.8) |
| Arthralgia | 1 | (4.5) | 4 | (18.2) | 4 | (20.0) | 9 | (14.1) |
| Diarrhoea | 3 | (13.6) | 1 | (4.5) | 5 | (25.0) | 9 | (14.1) |
| Gingival bleeding | 1 | (4.5) | 2 | (9.1) | 4 | (20.0) | 7 | (10.9) |
| Vomiting | 1 | (4.5) | 2 | (9.1) | 4 | (20.0) | 7 | (10.0) |
| Back pain | 1 | (4.5) | 2 | (9.1) | 3 | (15.0) | 6 | (9.4) |
| Dyspnoea | 2 | (9.1) | 3 | (13.6) | 1 | (5.0) | 6 | (9.4) |
| Nausea | | 0 | 2 | (9.1) | 4 | (20.0) | 6 | (9.4) |
| Pain in extremity | 2 | (9.1) | 2 | (9.1) | 2 | (10.0) | 6 | (9.4) |
| Petechiae | 3 | (13.6) | 1 | (4.5) | 2 | (10.0) | 6 | (9.4) |
| Platelet count increased | 2 | (9.1) | 2 | (9.1) | 2 | (10.0) | 6 | (9.4) |
| Upper respiratory tract infection | 2 | (9.1) | 1 | (4.5) | 3 | (15.0) | 6 | (9.4) |
| Dizziness | 1 | (4.5) | 1 | (4.5) | 3 | (15.0) | 5 | (7.8) |
| Insomnia | 1 | (4.5) | 3 | (13.6) | 1 | (5.0) | 5 | (7.8) |
| Asthenia | 3 | (13.6) | | 0 | 1 | (5.0) | 4 | (6.3) |
| Ecchymosis | | 0 | 2 | (9.1) | 2 | (10.0) | 4 | (6.3) |
| Abdominal pain upper | | 0 | | 0 | 3 | (15.0) | 3 | (4.7) |
| Constipation | | 0 | 3 | (13.6) | | 0 | 3 | (4.7) |
| Cough | 1 | (4.5) | | 0 | 2 | (10.0) | 3 | (4.7) |
| Migraine | | 0 | 1 | (4.5) | 2 | (10.0) | 3 | (4.7) |
| Mouth haemorrhage | | 0 | | 0 | 2 | (10.0) | 2 | (3.1) |
| Somnolence | | 0 | | 0 | 2 | (10.0) | 2 | (3.1) |

Table sorted in descending order by “total” column
Grouping method A was based on the mean daily dose received during the combined active treatment period, defined as the period between the day of first dose and the day of last dose of E5501 (excluding tapering period.
The Safety population included all subjects who received at least 1 dose of E5501 and had at least 1 posttreatment safety assessment in either the previous or current study.

Serious Adverse Events. Five subjects (1, 3, and 1 subjects in the lower, middle, and upper ⅓ dose level groups, respectively) reported 16 serious TEAEs (1, 9, and 6 cases in the lower, middle, and upper ⅓ dose level groups, respectively). In the upper ⅓ dose level group, all 6 serious TEAEs were reported by the same subject as shown in Table 21.

TABLE 21

| | Lower ⅓ | Middle ⅓ | Upper ⅓ | |
|---|---|---|---|---|
| | Mean Daily | Mean Daily | Mean Daily | |
| | Dose Level | Dose Level | Dose Level | E5501 |
| | (2.5-<8.85 mg) | (≧8.85-<13.5 mg) | (≧13.5-≦25.7 mg) | Total |
| MedDRA System Organ Class | (N = 22) | (N = 22) | (N = 20) | (N = 64) |
| Preferred Term | n (%) of subjects with: | | | |
| Any Serious TEAE | 1 (4.5) | 3 (13.6) | 1 (5.0) | 5 (7.8) |
| Blood and lymphatic system disorders | 1 (4.5) | 0 | 0 | 1 (1.6) |
| Thrombocytopenia | 1 (4.5) | 0 | 0 | 1 (1.6) |
| Cardiac disorders | 0 | 1 (4.5) | 1 (5.0) | 2 (3.1) |
| Mitral valve incompetence | 0 | 0 | 1 (5.0) | 1 (1.6) |
| Myocardial infarction | 0 | 1 (4.5) | 0 | 1 (1.6) |
| Gastrointestinal disorders | 0 | 1 (4.5) | 1 (5.0) | 2 (3.1) |
| Vomiting | 0 | 1 (4.5) | 1 (5.0) | 2 (3.1) |
| Diarrhoea | 0 | 0 | 1 (5.0) | 1 (1.6) |
| Nausea | 0 | 1 (4.5) | 0 | 1 (1.6) |
| General disorders and administration | 0 | 0 | 1 (5.0) | 1 (1.6) |
| site conditions | | | | |
| Chest pain | 0 | 0 | 1 (5.0) | 1 (1.6) |
| Pyrexia | 0 | 0 | 1 (5.0) | 1 (1.6) |
| Infections and infestations | 0 | 1 (4.5) | 0 | 1 (1.6) |
| Pneumonia | 0 | 1 (4.5) | 0 | 1 (1.6) |
| Investigations | 0 | 1 (4.5) | 0 | 1 (1.6) |
| Platelet count decreased | 0 | 1 (4.5) | 0 | 1 (1.6) |
| Nervous system disorders | 0 | 2 (9.1) | 1 (5.0) | 3 (4.7) |
| Cerebrovascular accident | 0 | 0 | 1 (5.0) | 1 (1.6) |
| Haemorrhage intracranial | 0 | 1 (4.5) | 0 | 1 (1.6) |
| Lethargy | 0 | 1 (4.5) | 0 | 1 (1.6) |
| Transient ischaemic attack | 0 | 1 (4.5) | 0 | 1 (1.6) |
| Vascular disorders | 0 | 1 (4.5) | 0 | 1 (1.6) |
| Pelvic venous thrombosis | 0 | 1 (4.5) | 0 | 1 (1.6) |

Grouping method A was based on the mean daily dose received during the combined active treatment period, defined as the period between the day of first dose and the day of last dose of E5501 (excluding tapering period.
The Safety population included all subjects who received at least 1 dose of E5501 and had at least 1 posttreatment safety assessment in either the previous or current study.

Recurrence of Thrombocytopenia. Recurrence of thrombocytopenia has been previously reported and is of special interest in this population. Recurrence of thrombocytopenia was defined as a platelet count that decreased to below 10,000/mm³ upon discontinuation of E5501. In this study, 9 subjects potentially met the criteria for recurrence of thrombocytopenia. Three were deemed serious; all 3 recovered.

Subjects who had recurrence of thrombocytopenia following discontinuation of study drug are shown in Table 22. All 9 cases occurred at an E5501 dose of 10 mg or above, and the majority (n=6/9) occurred within 2 weeks after discontinuation of E5501 (FIG. 3). However, recurrent thrombocytopenia was seen up to 4 weeks after the last dose of study drug.

TABLE 22

| | E5501 | Study Days | Platelet | | | |
|---|---|---|---|---|---|---|
| | Dose | (No. Days | Count | | Relation | |
| Subject | (mg) | Posttreatment^{a}) | (K/mm³) | Serious | To E5501^{b} | Outcome |
| A | 20-30^{c} | 193 (17) | 3 | No | Not related | Recovered |
| | | 235 (59) | 6 | No | Not related | Recovered |
| | | 249 (73) | 4 | No | Not related | Recovered |
| B^{d} | 10 | 209 (28) | 1 | No | Not reported | Unknown |
| C | 10-20^{c} | 182 (14) | 6 | No | Probably | Recovered |
| D | 20 | 193 (10) | 1 | Serious | Not related | Recovered |
| E^{d} | 10 | 209 (7) | 8 | No | Not reported | Unknown |
| F | 20 | 192 (11) | 8 | Serious | Not related | Recovered |
| G | 2.5-12.5^{f} | 179 (13) | 16^{g} | Serious | Probably | Recovered |
| H^{d} | 10-40^{h} | 143 (16) | 4 | No | Not related | Recovered |
| I | 10-20ⁱ | 181 (13) | 8 | No | Not related | Recovered |
| | | 233 (65) | 9 | No | Not related | Recovered |

Grouping method A was based on the mean daily dose received during the combined active treatment period, defined as the period between the day of first dose and the day of last dose of E5501 (excluding tapering period.
The Safety population included all subjects who received at least 1 dose of E501 and had at least 1 posttreatment safety assessment in either the previous or current study.
^{a}Study day is relative to the first dose of E5501 in the current study.
^{b}Relationship as assessed by the investigator
^{c}The subject received 20 mg through Day 113, then 30 mg from Day 114 until Day 176.
^{d}The subject's platelet count met the criteria for recurrent thrombocytopenia, but the investigator did not report the occurrence as an AE.
^{e}The subject received 10 mg through Day 86, then 20 mg from Day 87 until Day 168.
^{f}The subject received 2.5 mg through Day 41, then 12.5 mg from Day 42 until Day 166.
^{g}Platelet count as recorded 3 days before the event. No platelet count was recorded on the day of the event.
^{h}The subject received 10 mg through Day 14, 20 mg from Day 15 to Day 28, 30 mg from Day 29 to Day 79, and 40 mg from Day 80 until Day 127.
ⁱThe subject received 10 mg through Day 55, then 20 mg from Day 56 until Day 168.

Bleeding Events. During the combined study period, 43 (67.2%) of the 64 subjects reported a treatment-emergent bleeding event (Table 23). Gingival bleeding was the most frequently reported bleeding event, occurring in 7 subjects (10.9%). All were grade 1 (n=6) or grade 2 (n=1) and none were considered to be related to study drug. There did not appear to be a dose relationship.

Most subjects had episodes of bleeding that were grade 1 or grade 2. Four subjects experienced a clinically significant, grade 3 or 4 bleeding event as follows.
The first subject, a 50-year-old Asian man with no relevant history other than ITP, received E5501 2.5 mg once daily for 3 days in the previous study. Because of dizziness, the subject withdrew consent after 3 days' of treatment. That same day, he developed grade 4 gastrointestinal bleeding and was hospitalized (platelet count not reported at time of event, but was 2,000/mm³ 2 days earlier). Four days later, the event was considered resolved and the subject was discharged. The investigator deemed the event to be not related to E5501. Four days after discharge, the subject was readmitted with melantonic stool and general weakness (platelet count of 6000 [units not specified]). An esophagogastroduodenoscopy revealed grade 4 hemorrhagic diffuse gastritis. The event resolved on Study Day 17 (platelet count unknown). The investigator deemed the event to be not related to E5501, and stated that the subject had H. pylori, which may have contributed to the event.
The second subject, a 42-year-old white female with ITP and significant medical history of menorrhagia, received E5501 10 mg once daily in studies both studies. On Study Day 89, she presented with grade 3 intracranial bleeding (platelet count was 2,000/mm³ on Study Day 85). On Study Day 91, the subject underwent an emergency splenectomy. However, the subject's platelet count remained unstable and she required additional platelet transfusions. The subject continued to have grade 1 gum bleeding and epistaxis. Her last dose of study drug was on Study Day 100. The events resolved by Study Day 102 although her platelet count remained low (3,000 μL). The investigator classified all of the events as not related to study drug.
The third subject, a 44-year-old white female with ITP and history of splenectomy, received E5501 10 mg once daily in studies both studies. She completed treatment on Study Day 184, with a platelet count of 210,000/mm³. On Study Day 192, she developed grade 3 epistaxis which resolved 2 days later (platelet count unknown). The event was considered not related to study drug by the investigator. The subject's last visit was on Study Day 204, at which time her platelet count was 98,000/mm³.
The fourth subject, a 42-year-old white male with ITP and previous splenectomy, received double-blind E5501 5 mg once daily in the previous study and open-label E5501 10 mg once daily in the current study. The subject's platelet count remained at 4 to 6 K/mm³ (below the normal range) through Study Day 47. On Study Day 47, the subject experienced grade 3 increased bleeding (hemorrhagic diathesis) related to thrombocytopenia and E5501 was permanently discontinued. At that time, his platelet count was 4 K/mm³. Despite treatment with methylprednisolone 40 mg, acetaminophen 650 mg, diphenhydramine 50 mg, immunoglobulin 42 g and platelets 1 unit (each one time only), the bleeding had not resolved as of Study Day 80. The investigator classified the subject's bleeding as not related to E5501.

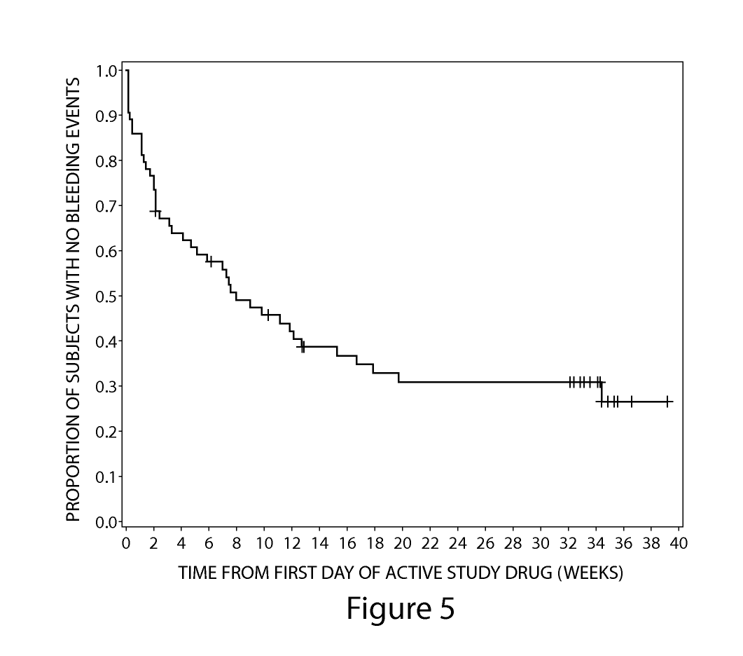

Using Kaplan-Meier methods, the estimated median time to the first bleeding event was 8 weeks (95% CI: 4.7-15.3 weeks) after initiation of E5501 (FIG. 5).

TABLE 23

| | Lower ⅓ | | Middle ⅓ | | Upper ⅓ | | |
|---|---|---|---|---|---|---|---|
| | Mean Daily | | Mean Daily | | Mean Daily | | |
| | Dose Level | | Dose Level | | Dose Level | E5501 | |
| | (2.5-<8.85 mg) | | (≧8.85-<13.5 mg) | | (≧13.5-≦25.7 mg) | Total | |
| | (N = 22) | | (N = 22) | | (N = 20) | (N = 64) | |
| Severity (Toxicity Grade) | n (%) of subjects with: | | | | | | |
| Any TE bleeding event | 15 | (68.2) | 14 | (63.6) | 14 (70.0) | 43 | (67.2) |
| Grade 1 | 11 | (50.0) | 9 | (40.9) | 8 (40.0) | 29 | (45.3) |
| Grade 2 | 2 | (9.1) | 3 | (13.6) | 6 (30.0) | 11 | (17.2) |
| Grade 3 | 1 | (4.5) | 2 | (9.1) | 0 | 3 | (4.7) |

| | | | | | | |
|---|---|---|---|---|---|---|
| Grade 4 | 1 | (4.5) | 0 | 0 | 1 | (1.6) |

| | | | | |
|---|---|---|---|---|
| Grade 5 | 0 | 0 | 0 | 0 |

NCI-CTCAE = National Cancer Institute Common Toxicity Criteria for Adverse Events; TE = treatment-emergent
If a subject had more than 1 bleeding event, the event with the highest toxicity grade was counted. Grouping method A was based on the mean daily dose received during the combined active treatment period, defined as the period between the day of first dose and the day of last dose of E5501 (excluding tapering period).
The Safety population included all subjects who received at least 1 dose of study drug and had at least 1 posttreatment safety assessment in either the previous or current study.

Thrombolytic Events. Two of these 3 subjects reported their thrombotic events during Week 10 of treatment. One subject, a 73-year-old white female who was receiving E5501 10 mg, had grade 3 deep vein thrombosis (DVT) in the iliac vein on Study Day 69, considered not related to study drug. At the time of the event, her platelet count was 19,000/mm³. Treatment with E5501 was permanently discontinued and the event resolved. The subject had previous breast cancer and factor V Leiden mutation as risk factors. The second subject, a 44-year-old white female, had a grade 3 stroke. She had been receiving E5501 30 mg and had a platelet count of 119,000/mm³ at the time of the event. Treatment with E5501 was interrupted and the event resolved. This subject also had the following risk factors: ANA+, lupus coagulant+, and heterozygote for factor V Leiden. The third subject, a 41-year-old white female who was receiving E5501 20 mg, had grade 1 superficial thrombophlebitis on Study Day 51, considered not related to study drug. The event resolved.

The fourth subject had a thromboembolic event in the previous study. This subject, a 71-year-old white male, had a transient ischemic attack (TIA), myocardial infarct (MI), and pneumonia after 20 days of once daily treatment with E5501 10 mg. An ECG demonstrated normal sinus rhythm with a ventricular rate of 94 beats per minute (bpm), ST and T wave abnormalities consistent with lateral ischemia, and serial changes of inferior infarct when compared with a previous ECG recorded on Study Day 8. The physician felt that the TIA was a new small, left parietotemporal stroke, and simultaneously associated with a subacute MI, may have been an embolic event. At that time, his platelet count was 47 K/mm³. Treatment with E5501 was permanently discontinued and the events resolved. The investigator deemed the MI and TIA to be possibly related to E5501 and the pneumonia to be not related to E5501. After withdrawal of E5501, the subject developed serious, grade 4 retinal artery occlusion on Study Day 36 (14 days posttreatment). At that time, his platelet count was 27 K/mm³. This event had not resolved as of the last report date. The investigator deemed this event to be possibly related to E5501. This subject had a significant medical history of TIAs, MIs, status post-coronary artery bypass graft (s/p CABG), and angioplasty.

Neoplasms. Three neoplastic events of interest occurred during this study. One subject (125-5063) who was receiving E5501 10 mg experienced mild leukocytosis during the first study. On Day 8 of the present study, the subject's white blood cell count was 28.8 K/L and he underwent a bone marrow biopsy and aspirate, which revealed myeloid hyperplasia with left-shift and increased blasts (11%). Chromosome analysis revealed trisomy 8 in 40% of metaphase cells and the subject was diagnosed with myelodysplastic syndrome (REAB classification). A peripheral blood smear exhibited 10% blasts. The subject was diagnosed with myelodysplastic syndrome (MDS) and study drug was discontinued on Study Day 9. The leukocytosis worsened to grade 3 by Day 23 of the current study, at which time the subject was permanently withdrawn from the study. A diagnosis of acute myelogenous leukemia (AML) was made on Study Day 57. The subject was treated with chemotherapy for the AML and recovered with sequelae as of the last follow-up. The investigator considered the leukocytosis to be probably related to study drug and the AML to be possibly related to study drug. Because no bone marrow biopsy was taken at baseline, the presence of pre-existing MDS cannot be ruled out.

A second subject, who was receiving E5501 10 mg, had a grade 2, benign lipoma, located on his back, on Study Day 35. The AE was considered not related to study drug and the subject recovered.

The third subject was receiving E5501 20 mg and had grade 2 myeloproliferative disorder on Study Day 15, considered not related to study drug. At the onset of the event, the subject had substantial splenomegaly, but a PET scan was negative and the subject had no increase in blast count in a peripheral blood smear nor in a bone marrow biopsy. The bone marrow biopsy showed that the marrow was normocellular for the subject's age. Megakaryocytes were markedly increased. These were predominantly of large size with dysplastic features and frequently arranged in clusters. Because E5501 is a thrombopoietin agonist, these features may be indications that the study drug was having a positive effect on the pathway for platelet synthesis. The subject was lost to follow up and the outcome is unknown.

Liver Toxicity. Two subjects in the safety population experienced a grade 2 or 3 elevation in AST and ALT concentration. One subject, was a 69-year-old female with ITP and a history of hypertension, osteoporosis, high cholesterol, and kidney stones who had been receiving E5501 20 mg/day in the previous study with no abnormalities in liver function tests (LFT) during the study. Approximately 164 days after initiation of E5501, she experienced AEs consisting of grade 3 elevations in AST (431 U/L) and ALT (421 U/L). The LFT concentrations decreased over the next 2 months, completely resolving while the subject was still receiving E5501 20 mg/kg. The events were considered not related to E5501 by the investigator and were confounded by the concomitant use of 3 drugs that are associated with abnormalities of liver function: Crestor®, ibuprofen and prednisone. The ibuprofen had been initiated about 3 months prior to the onset of the LFT elevations.

The second subject was a 22-year-old female with ITP who was receiving E5501 5 mg/day. Approximately 21 days after the initiation of E5501 treatment, she experienced asymptomatic, grade 2 elevations in serum AST (143 U/L) and ALT (210 U/L) concentrations on standard laboratory testing. These elevations were not associated with increases in total bilirubin or alkaline phosphatase. These elevations completely recovered while the subject was maintained on E5501 without change in dose within 2 weeks. Confounding factors included concomitant use of 1 dose of acetaminophen approximately 18 days before the event. The subject was considered a responder and continued to receive E5501 5 mg/day in the current study, during which time the subject had 2 separate episodes of isolated grade 1 elevations in ALT at 126 and 186 days after initiation of E5501. The event was considered possibly related to study drug by the investigator.

### Discussion

As might be expected in this population, the incidence of TEAEs was high. Nearly all subjects (n=62/64, 96.9%) who received E5501 treatment experienced one or more TEAEs. Most events were mild in severity, transient, and completely resolved. The incidence of several AEs, e.g., nausea, dizziness and headache (including migraine), was highest at the upper ⅓ mean daily dose level, suggesting a possible dose-response relationship. However, the overall incidence of TEAEs was similar across the 3 mean daily dose levels. Moreover, no difference in the incidence of serious AEs, AEs leading to premature discontinuation, or any AEs of special interest was observed across the 3 mean daily dose levels. Note that any meaningful interpretation of dose level on the incidence of AEs is confounded by the method of dose assignment in this study; e.g., based on responder status with dose adjustments based upon self-selection rather than a random assignment.

The safety profile of E5501 was also characterized by a low incidence of serious AEs or AEs that led to interruption or premature discontinuation of E5501. Compared with the 4-week safety profile of E5501, the additional 6-month safety results were consistent and remained favorable. Increased platelet count, especially at levels exceeding 450,000/mm³, could potentially lead to thromboembolic complications. Risk factors include a history of thrombotic events, presence of antiphospholipid antibodies, and factor V Leiden mutation. In both studies combined, thromboembolic events occurred in 4 subjects (approximately 6% of treated subjects). All 4 subjects had been receiving E5501 doses of 10 mg/day or higher. Three of these 4 subjects had clear risk factors. Two of the 4 subjects had at least 2 risk factors, including factor V Leiden mutation. A third subject had a significant history of TIA, MI, s/p CAGB, and angioplasty. The fourth subject, who had grade 1 superficial thrombophlebitis, had no reported pre-existing risk factors. At the onset of the thrombophlebitis, this subject's platelet count was high (571,000/mm³). However, the AE resolved in 7 days, despite her platelet count remaining elevated.

The frequency of thromboembolic events in the combined E5501 studies is consistent with literature data indicating that the incidence of thromboembolic events in patients with ITP ranges from 4% to 6%. These results suggest that the thromboembolic events are more likely related to the subjects' underlying disease rather than to E5501 in the previous and current study. However, because the sample size of the individual dose groups was small and the current study lacked a placebo control group, an association with E5501 treatment cannot be entirely ruled out.

Liver toxicity may range from mild, asymptomatic elevations in LFTs to frank drug-induced liver injury. In the current study, 2 subjects had LFT elevations. Both subjects had LFT elevations that were asymptomatic and transient. For 1 subject, the LFT elevations resolved while the subject was receiving stable doses of E5501. The second subject had multiple episodes of LFT elevations which were all mild. Based on the transience and mildness of these events, the presence of other confounding factors like the use of concomitant statins or nonsteroidal antiinflamatory drugs, and the resolution of the events while the subjects continued to receive E5501, a causal relationship appears unlikely. Although a causal relationship cannot be completely excluded, the current evidence does not support E5501 as having a medically important hepatotoxic effect.

Recurrent thrombocytopenia occurred in 9 subjects at E5501 doses of 10 mg/day and above. Some of the events were serious. Recurrent thrombocytopenia may be prevented by tapering the dose of E5501 rather than withdrawing drug abruptly.

The results of this study demonstrate that E5501 not only increased platelet counts in the shortterm, but also maintained the platelet response over a 6-month period as measured by durable response rates for 52.8% (FAS) and 75% (SE) of subjects and overall platelet response rate for 75.5% (FAS) and 94.4% (SE) of subjects. The study also provided information about the time-course of platelet response over a 6-month treatment period and showed that platelet response occurred early during treatment and median platelet counts in the FAS were able to be maintained above 50,000/mm³ throughout the study.

In the current study, 49% (n=26/53) of subjects needed an upward dose titration. However, only one-half of these subjects (n=13/26) received it. When subjects received a dose titration, responders in the previous study continued to have an overall response in 100% of cases while non-responders in the previous study improved their overall response rate in 87.6% of cases. Ultimately, the median platelet count in the SE population increased. Moreover, subjects who were receiving low doses of study drug (e.g., placebo, E5501 2.5 mg, or E5501 5 mg) in the previous study and had not responded to those dose levels did respond when offered a higher, open label starting dose of E5501 10 mg in this study. The overall response rates in these non-responders (70%-80%) were comparable to the response rate (71.4%) in subjects who had responded to E5501 10 mg in the previous study and continued to receive that dose in the current study.

Five subjects did not respond to E5501 10 mg in the previous study. Two of these subjects responded to E5501 in this study when their dose was increased to 20 mg.

Three of the 5 subjects remained no-nresponders in this study. Two of these subjects continued to receive E5501 10 mg in the current study despite requiring an upward dose titration. The third subject who did not meet platelet response criteria had widely fluctuating platelet counts (>1,000,000/mm³ and <10,000/mm³) upon multiple dose adjustments of E5501. These results suggest that dose adjustments are probably needed for individual subjects with ITP. Moreover, subjects who did not respond to low doses of E5501 may not necessarily have an inherent resistance to study drug and may actually respond when treated with higher doses of E5501.

Twenty-four of 53 subjects achieved a reduction in their need for steroids. Thirteen of these 4 subjects were able to decrease their steroid dose by at least 50%. One-third of the 24 subjects were able to permanently discontinue their use of steroids. Collectively, the data in the current study indicate that E5501 is an effective treatment for chronic ITP, and the efficacy findings are consistent with the observations in the previous study.

### Conclusion

Based on the results of this study, E5501 was well tolerated and demonstrated a favorable safety profile over a 6-month treatment period. E5501 also demonstrated effectiveness as measured by durable and overall platelet response rates, as well as reduction or withdrawal of concomitant steroid medications, in subjects with chronic, refractory ITP. The majority of responders in the previous study maintained their platelet response in the current study, while continuing to receive their initial dose throughout the 6-month treatment period. The majority of non-responders in the previous study who received upward dose titration in this study responded to E5501 at a higher dose. Both safety and efficacy data from this 6-month extension study are consistent with the results of a previous 28-day study and support the conclusions from that study

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The contents of all references, patents and patent applications cited throughout this application are hereby incorporated by reference.

1. A method for rapidly increasing a platelet response in a subject at risk of bleeding due at least in part to a low platelet count, the method comprising:
administering to a subject with a low platelet count an effective amount of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid,
wherein the platelet response is increased in less than 14 days, such that the subject is at decreased risk for bleeding.

2. The method of claim 1, further comprising the step of detecting the platelet response within about 14 days, about 7 days, about 3 days or about 24 hours after administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid.

3. The method of claim 1, wherein the subject has an initial platelet count of less than or about equal to 30,000/mm³ and the platelet count is increased in less than about 7 days to greater than or equal to about 50,0000/mm³.

4. The method of claim 1, wherein the subject has a response rate at about 28 days of at least about 10%, about 25%, about 50% or about 80% over an initial platelet count and compared to a subject being administered a placebo.

5. The method of claim 1, wherein the subject has a response rate at about 7 days of at least about 5%, about 10%, about 25%, about 50%, about 70%, about 90% or about 98% over an initial platelet count and compared to a subject being administered a placebo.

6. The method of claim 1, wherein the subject has a sustained platelet response of at least about 25%, about 30%, about 40%, about 50%, about 75% or about 77%.

7. The method of claim 1, wherein the platelet response is sustained for at least about 7 days, about 14 days, about 28 days, about 2 months, about 3 months, about 4 months, about 5 months or about 6 months.

8. The method of claim 1, wherein the platelet response is increased by at least about 10%, by about 25%, by about 50% or by about 90% over an initial platelet count.

9. The method of claim 1, wherein the platelet response is increased (net change or achieved) by between at least about 10,000/mm³ and about 400,000/mm³.

10. The method of claim 1, wherein said effective amount is an effective periodic dose.

11. The method of claim 13, wherein said effective periodic dose is a once daily dose, a twice daily dose, a thrice daily dose, a dose administered every other day, a weekly dose or a monthly dose.

12. The method of claim 13, wherein the effective periodic dose is administered for about 1 month, about 2 months, about 3 months, about 4 months, about 5 months, about 6 months, or about 7 months.

13. The method of claim 1, wherein the subject is in need of treatment that may induce bleeding.

14. The method of claim 16, wherein the subject has a low platelet count within at least about 1 month of treatment, within at least about 14 days of treatment, within at least about 7 days of treatment or at least about the time of treatment.

15. The method of claim 16, wherein the treatment comprises a surgery, chemotherapy or radiation therapy or a combination thereof.

16. The method of claim 15, wherein the surgery comprises administration of an anesthetic, a biopsy, administration of an epidural, a transplantation or a dental procedure.

17. The method of claim 1, wherein the subject has thrombocytopenia.

18. The method of claim 17, wherein the subject further is in need of treatment for cancer, liver disease, vitamin B12 deficiency, a systemic viral infection, a systemic bacterial infection, sepsis, dengue fever or an immune disorder.

19. The method of claim 18, wherein the liver disease is chronic viral hepatitis, nonalcoholic steatohepatitis, alcoholic liver disease, liver failure or sepsis.

20. The method of claim 17, wherein the thrombocytopenia is chronic immune (idiopathic) thrombocytopenic purpura, radiation-induced thrombocytopenia, chemotherapy-induced thrombocytopenia, HIV/AIDS-induced thrombocytopenia, anemia-induced thrombocytopenia, thrombotic thrombocytopenic purpura or neonatal alloimmune thrombocytopenia.

21. The method of claim 1, wherein the subject is likely to develop thrombocytopenia due to chemotherapy or radiotherapy and 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid is administered prophylactically such that the subject is at decreased risk for bleeding.

22. The method of claim 1, wherein said subject has a durable platelet response.

23. The method of claim 1, wherein said subject has a transient platelet response.

24. The method of claim 1, wherein said subject maintains the platelet response.

25. The method of claim 1, wherein said subject has had less than 3 lines of prior therapy.

26. The method of claim 1, wherein said subject has had 3 or more lines of prior therapy.

27. The method of claim 1, wherein said subject has a history of splenectomy.

28. The method of claim 1, wherein said subject has no history or splenectomy.

29. The method of claim 1, wherein said subject concomitantly uses steroid medications.

30. The method of claim 29, wherein said steroid is prednisone.

31. The method of claim 29, wherein said subject reduces steroid use upon administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid.

32. The method of claim 29, wherein said subject permanently discontinues said steroid use upon administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid.

33. The method of claim 6, wherein the platelet response is sustained for about 1 week, about 2 weeks, about 3 weeks or about 4 weeks after discontinuation of administration of 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid.
